(19) United States
(12) Patent Application Publication
(10) Pub. No.: US 2023/0031007 A1
(43) Pub. Date: Feb. 2, 2023

(54) METHODS AND APPARATUS FOR REMOVAL OF SKIN PIGMENTATION AND TATTOO INK

(71) Applicant: LIGHTSENSE ISRAEL LTD., Ra'anana (IL)

(72) Inventors: Dan Dov GROSSMAN, Herzliya (IL); Oded LOEBL, Tel Mond (IL); Guy ENGEL, Herzliya (IL); Errol DAMELIN, London (GB); Jaren Aron EISENBERG, London (GB)

(21) Appl. No.: 17/885,669

(22) Filed: Aug. 11, 2022

Related U.S. Application Data
(62) Division of application No. 17/132,706, filed on Dec. 23, 2020, now abandoned.
Application No. 17/132,706 is a continuation of application No. PCT/IB2019/055368, filed on Jun. 25, 2019.

(30) Foreign Application Priority Data
Jun. 27, 2018 (GB) 18100495.0
Jun. 27, 2018 (GB) 1810496.8
Jul. 10, 2018 (GB) 1811297.9

(51) Int. Cl. A61B 18/20; A61B 34/30; A61B 90/50

(52) U.S. Cl. CPC A61B 18/203; A61B 34/30; A61B 90/50; A61B 2090/364

(57) ABSTRACT

Methods and apparatus for dermatological laser treatment, e.g. for the removal of unwanted tattoos or other skin pigmentation. Removal of multiple colors with a single pulsed laser beam may be achieved using intensities in excess of about 50 GB/cm². Methods for reducing the pain and tissue damage associated with laser tattoo removal include using a spot size of less than 2 mm with a fluence in the range of 0.5-10 J/cm². Scanning the laser beam over an area of skin to be treated allows such areas to be treated accurately with scanning patterns calculated to promote rapid dissipation of heat away from treated portions of the skin. Multiple treatment rooms may be served by a single pulsed treatment laser by beam toggling, splitting or pulse-picking to minimise downtime of the laser.

### CROSS-REFERENCE TO RELATED APPLICATIONS

This is a divisional of U.S. Application No. 17/132,706, filed Dec. 23, 2020, which is a continuation of international Application No. PCT/IB2019/055368, filed on Jun. 25, 2019, which claims priority to GB Applications Nos. GB1810495.0, filed on Jun. 27, 2018; GB1810496.8, filed on Jun. 27, 2018; and GB1811297.9, filed on Jul. 10, 2018, the entire contents of each of which being fully incorporated herein by reference.

### FIELD OF THE INVENTION

The present invention relates to the removal of unwanted skin pigmentation and tattoo ink and comprehends various improvements in and relating to methods and apparatus for the same.

### BACKGROUND TO THE INVENTION

Tattooing and other pigmentation of the skin involves the placement of pigment into the skin’s dermis, i.e. a layer of dermal tissue underlying the epidermis which is typically about 2 mm thick. After initial injection, pigment is dispersed throughout a homogenised, damaged layer down through the epidermis and upper dermis, in both of which the presence of foreign material activates the immune system’s phagocytes to engulf the pigment particles. As healing proceeds, the damaged epidermis flakes away (eliminating surface pigment) while deeper in the skin granulation tissue forms, which is later converted to connective tissue by collagen growth. This mends the upper dermis, where pigment remains trapped within successive generations of macrophages, ultimately concentrating in a layer just below the dermis/epidermis boundary at a depth below skin surface of between about 300-700 µm. Its presence there is stable, but in the long term (decades) the pigment tends to migrate deeper into the dermis, accounting for the degraded detail of old tattoos. According to a Harris Poll in 2016 (Shannon-Missal, 2016), almost half of people between 18 and 35 in the United States have tattoos and almost one in four regrets it. Based on an estimate of about 60 million people in that age group, this means that about 7.5 million people have “tattoo regret”. Accordingly, many people with tattoos seek to have them removed.

At the time of the present application, the gold standard modality for tattoo removal is non-invasive removal of tattoo pigments using Q-switched lasers. The application of radiation to a subject’s skin is done manually: an operator points a laser beam at an area to be treated and fires the laser. Different types of Q-switched lasers are used to target different colours of tattoo ink depending on the specific light absorption spectra of the tattoo pigments. Typically, black and other darker-coloured inks can be removed completely using Q-switched lasers while lighter colours such as yellows and greens are very difficult to remove. Success can depend on a wide variety of factors including skin colour, ink colour, and the depth at which the ink was applied.

Pulsed laser treatment is also used to remove unwanted skin pigmentation and pigmented lesions, such as freckles, age-spots, sun-spots, liver-spots, melasma and superficial vascular malformations such as port wine stains and telangiectasias.

Laser removal of unwanted pigmentation is typically carried out using skin surface input intensities of about 10⁹-10¹⁰ W/cm² and pulse widths of greater than 250 ps to achieve fluences of about 1-7 J/cm². To yield these input fluences, pulse energies in the range 200-800 mJ are used in conjunction with 2-6 mm spot sizes on skin surface and ~1-10 Hz pulse repetition rates.

Such methods are colour selective, it being assumed the main interaction of laser with tissue, mainly absorption, is solely a function of wavelength with respect to target pigment absorption. Indeed, this is the main idea behind selective photothermolysis (Anderson & Parrish, 1983). It is widely thought that there must be a match between the laser radiation wavelength and the colour of the ink or lesion to be removed. For example, it is well known that red ink can be removed by a green (532 nm) laser, while a green ink requires an infra-red (IR) (800 nm) wavelength. When a mismatch occurs (i.e. when an unsuitable laser wavelength is used), laser radiation is not absorbed by the target colour and no removal is achieved. This is especially problematic when a single tattoo has several colours that require different laser wavelengths. Current high-end laser removal systems offer a variety of wavelength lasers to accommodate different tattoo colours, but with limited success and high cost. The number of treatments required can range from eight to as many as 20, lasting between one and two years.

An object of the present invention therefore is to provide methods and apparatus for removal of skin pigmentation or tattoo ink that can remove multiple different colours using laser light of a single wavelength.

Current tattoo removal treatment systems suffer from several other drawbacks (Goldman, Fitzpatrick, Ross, Kilmer, & Weiss, 2013):
Pain is an integral part of the treatment and local anaesthetics are usually applied.
Damage to skin is intensive. Immediate side effects such as swelling, tenderness, pinpoint bleeding, blistering and itching are considered a normal, immediate result of treatment. Adverse events such as acute pain, limb oedema, frank bleeding, bulla and intractable pruritus can sometimes occur. Scarring, textual changes and hypertrophic scarring are other possible and common complications which may be acute or chronic.
Owing to extensive damage, recovery time between treatments is long, at least six weeks or more are required for complete tissue recovery.

The above-mentioned disadvantages are considered as major barriers for the wide spread adoption of prior art systems for pigment removal.

Another object of the present invention therefore is to reduce the amount of pain and skin damage experienced by a subject when they undergo laser treatment for the removal of unwanted skin pigmentation.

Yet another object of the invention is to shorten recovery times between successive laser treatments to allow for more rapid removal of unwanted skin pigmentation.

Further, the manual application of a laser to a subject’s skin is inherently slow and/or inaccurate. Although an aiming beam is sometimes used to provide positional feedback for the operator, it is hard to maintain accurate performance and high throughput. When an area to be treated has small features and sizes, e.g. an intricate tattoo or a group of small lesions, the beam size further hampers accuracy of laser treatment. Even using the smallest current beam size in these applications of about 2 mm, it is both time consuming and inaccurate to treat areas of features smaller than 2 mm. Moreover, the accuracy of placement depends entirely on the operator’s expertise, dexterity, experience and patience, all of which can vary considerably. While covering large pigmented areas, operators typically use a pulsed laser with a pulse rate of 10 Hz and moves the beam very quickly across the area. This is an intrinsically inaccurate process and involves a high degree of inevitable overlapping of successive pulses on the skin and concomitant additional damage to the tissue. It is clear therefore that manual placement of laser radiation is inconsistent, inaccurate and time consuming and may exacerbate damage to the skin that is occasioned by such prior laser treatment methods.

Yet another object of the present invention therefore is to provide methods and apparatus for the removal of tattoos and other unwanted localised skin pigmentation that is more accurate than previous methods, leading to reduced treatment times and/or reduced tissue damage.

Yet another object of the invention is to provide methods and apparatus for testing dermatological laser treatment apparatus to check that it will operate consistently and reliably within predetermined, safe operating parameters.

Further objects of the invention will apparent from the following description of the invention, particularly from its technical advantages over existing methods of removing unwanted pigmentation from the skin.

As will be apparent, there are multiple different aspects of the present invention, which may be used together or separately as desired by those skilled in the art. The various different technical problems associated with prior laser-based methods of dermatological treatment that are discussed above are addressed by different aspects of the present invention. It is not intended that each separate aspect of the invention will necessarily solve all of the above-mentioned problems on its own.

### SUMMARY OF THE INVENTION

It has now been surprisingly found that laser interaction with a pigment or tattoo ink in skin is not only a function of wavelength, but rather a function of wavelength and/or pulse duration and/or intensity. In particular, it has been found that increasing the intensity of the laser light to the level of about 10¹¹-10¹² W/cm² with fluence values that are similar to those used in the prior art, i.e. about 1-7 J/cm² may procure efficient removal of different colours using a single laser wavelength. It has been found that the intensity of the laser light needs to be high enough so as to interact significantly with all colours, particularly visible colours. As opposed to a sharp threshold of non-linear optical break-down, it has been found that for pigment in live skin there is a wide range of intensities that can be used for interacting with multiple colours using the same wavelength.

In a first aspect of the present invention therefore there is provided a method of dermatological treatment comprising irradiating an area of a subject’s skin to be treated with a pulsed beam of laser light; characterised in that the laser light has an intensity of at least about 50 GW/cm² and a pulse width in the range of about 0.1-100 ps.

Suitably, in some embodiments, the pulsed laser light may be produced by a mode-locked laser of the kind known to those skilled in the art.

Within each treatment, the area of the subject’s skin to be treated is usually irradiated only once. Typically, the area to be treated is larger than a spot of laser light where each pulse is incident on the subject’s skin. Thus, to treat the whole area, the beam may be moved over the area, either by moving the beam itself or the area to be treated, as described in more detail below, so that successive pulses fall on separate portions of the area, each portion having a size that is substantially the same as the size of the spot of laser light produced by the beam. Each portion of the area receives one pulse of the laser light within a single treatment. The separate portions of the area of skin that are irradiated by the pulses within a single treatment are preferably non-overlapping with one another.

Suitably, the pulsed laser light may have a fluence in skin depth of about 0.5-10 J/cm². In some embodiments, the fluence in skin depth may be about 1-8 J/ cm² or 1-7 J/cm². By ‘skin depth’ herein is meant the depth at which pigment is typically located in the dermis, i.e. about 200-1000 µm below the surface of the skin (epidermis).

In some embodiments of the invention, the dermatological treatment may be purely cosmetic and may consist, for example, in removing skin pigmentation or tattoo ink or treating other cutaneous conditions for non-medical purposes. Cutaneous conditions that may be treated in accordance with the present invention include vascular lesions including superficial vascular malformations (port-wine stains), facial telangiectasias, haemangiomas, pyogenic granulomas, Kaposi sarcoma and poikiloderma of Civatte.

- Pigmented lesions including freckles and birthmarks including some congenital melanocytic naevi, blue naevi, naevi of Ota/Ito and Becker naevi.

- Facial wrinkles, acne scars, keloids and hypertrophic scars, and sun-damaged skin

However, in some embodiments, the methods of the invention may be used for medical purposes. In particular, the methods of the invention may be used for medical treatment of cutaneous conditions such, for example, acne, inflammatory dermatoses, benign and malignant cutaneous tumours.

By “removal” of skin pigmentation, tattoo ink or other cutaneous conditions is meant removal in whole or in part. Typically, a lesion or tattoo will not be removed by a single treatment, but the intensity of its colouration will be reduced. Complete removal may require a plurality of successive treatments over a period of time with the colour becoming progressively lighter after each treatment, as described in more detail herein. Full clearance of colour from the skin (to the naked eye) may be achieved after a finite number of sessions. The number of sessions might vary from one target colour to the next, but in any case, the number of sessions from one colour to the next will not vary by more than a factor of about two. As described in more detail below, an advantage of the methods of the present invention is the ability to carry out treatments more frequently on a given individual to shorten the total time for complete removal of the lesion or ink.

The use of high intensity pulsed laser light in accordance with the invention may allow for a range of colours of pigment or ink, particularly visible colours, to be removed using a single wavelength of light. This contrasts sharply with previous methods in which the wavelength of the laser is closely matched to the colour of a lesion or tattoo to be removed. In some embodiments of the invention, the intensity of the laser light may be selected for a given wavelength and fluence to obtain removal of at least three different colours of pigment or lesion. The colours may , for example, be selected from black, green, yellow, red and orange. Advantageously, the intensity of the laser may be selected to obtain removal of multiple colours including, for example, purple and pink. For a given wavelength, the intensity of laser light required to remove a plurality of selected colours may be selected empirically by measuring the reaction threshold of different ink colours/skin pigments while increasing the intensity of the laser light at a constant fluence. By finding the highest intensity needed to remove a “worst case” colour, i.e. the colour that is most difficult to remove, a suitable working intensity may be identified that covers all of the selected colours.

In some embodiments, the working intensity of the laser light may be selected to introduce enough intensity so that all target colours become absorbing/absorbent owing to similar non-linear processes of absorption.

Thus, in some embodiments, the laser light may have an intensity of about 0.1-1 TW/cm².

In accordance with a particular aspect of the present invention therefore, there is provided a method of dermatological treatment comprising irradiating an area of a subject’s skin to be treated with a pulsed beam of laser light; characterised in that the laser light has an intensity of at least about 50 GW/cm².

Suitably, the laser light may have a pulse width of at least about 0.5 ps, preferably at least 1.0 ps. In some embodiments, the laser light may have a pulse width of less than about 35 ps, preferably less than about 25 ps. Thus, the laser light may have a pulse width in the range about 1-15 ps, preferably about 1-10 ps.

In accordance with a different aspect of the present invention therefore, there is provided a method of dermatological treatment comprising irradiating an area of a subject’s skin to be treated with a pulsed beam of laser light; characterised in that the laser light has a pulse width in the range of about 0.1-100 ps.

Advantageously, the laser light may have a spot size on the skin of less than about 2 mm in diameter. In some embodiments, the laser light may have a spot size of about 0.1-1.5 mm; typically about 0.5-1.0 mm. Such a spot size is smaller than the spot sizes of 2-6 mm used in previous methods. For the same fluence, a smaller spot size will necessarily result in less energy being input to the skin as compared with a larger spot size. However, for a given fluence, on a somewhat simplified analysis, a pulse of laser light will produce substantially the same increase in temperature within a volume of skin irradiated by the laser light regardless of spot size. (Owing to interaction of complicated tissue structure and light, there can be small volumes of higher elevated temperatures). For instance, a laser pulse having a fluence of about 2.5 J/cm² will result in a temperature rise of about 15° C. regardless of whether a spot size of 5 mm or 0.5 mm is used. An advantage of using a small spot size of relatively low energy is that the temperature of the skin will fall more rapidly after irradiation as compared with a larger spot of relatively high energy.

It is known that when skin is subjected to an elevated temperature, there is an inverse relationship between the elevated temperature the skin can withstand without tissue damage (e.g. coagulation) and the length of time the skin is subjected to the elevated temperature. Studies have shown that surface temperatures of 44° C. do not produce burns unless exposure time exceeds about 6 hours. At temperatures in the range 44° C. to 51° C., the rate of epidermal necrosis approximately doubles with each half degree Celsius. At 70° C. or more, the exposure time required to cause transepidermal necrosis is less than 1 s (Pierce County Emergency Medical Services). These numbers represent an external heat source applied to the skin. In the case of laser radiation heating tissue from the inside owing to embedded pigment absorption, these numbers are likely to be optimistic and damage may occur sooner.

Suitably therefore, the fluence of each pulse of laser light incident on the subject’s skin and the spot size should be controlled such that the fluence falls within the range of about 0.5-10 J/cm² and the spot size is such that the skins cools down sufficiently rapidly after irradiation that the skin is not subjected to an elevated temperature greater than 44° C. for longer than the threshold duration at which damage to the skin results at the elevated temperature.

Thus, in accordance with a second aspect of the present invention, there is provided a method of dermatological treatment comprising moving a pulsed beam of laser light over an area of a subject’s skin to be treated; wherein each pulse impinges on a different portion of the subject’s skin within the area to be treated in the form of a spot and the fluence of each pulse in skin depth is in the range of about 0.5-10 J/cm²; characterised in that the size of each spot is such that the skins cools down sufficiently rapidly that the skin is not subjected to an elevated temperature greater than 44° C. for longer than the threshold duration at which damage to the skin results at the elevated temperature.

Suitably, the fluence of each pulse in skin depth may be in the range of about 1-8 J/cm²; or 1-7 J/cm², as mentioned above.

Suitably, the size of the spot may be such that the thermal relaxation time of the skin (which may be defined as the time taken for the temperature of the skin to fall by one half of its initial temperature rise) is shorter than the length of time for which the skin can withstand the initial temperature rise before damage to the skin is caused. By ‘initial’ temperature rise in this context is meant the maximal temperature reached by the skin following receipt of a pulse of laser light. Typically, the thermal relaxation time may be in the range of about 0.1 s to about 8 s. For example, in some embodiments, the size of the spot produced by the laser light on the subject’s skin should be such that the temperature of the skin does not rise above about 51° C. and affords a relaxation time or no more than about 6 s.

As mentioned above, in some embodiments, the spot may have a diameter of less than about 2 mm. Generally, the spot may be circular, but in other embodiments, the spot may have a different shape with a maximum dimension of less than about 2 mm.

Thus, in accordance with a third aspect of the present invention, there is provided a method of dermatological treatment comprising moving a pulsed beam of laser light over an area of a subject’s skin to be treated; wherein each pulse impinges on a different portion of the subject’s skin within the area to be treated in the form of a spot and the fluence of each pulse in skin depth is in the range of about 0.5-10 J/cm²; characterised in each spot has a maximum dimension of less than about 2 mm.

As described above, each spot may suitably have a maximum dimension of 1.5 mm or 1 mm.

The fluence of each pulse in skin depth may be in the range of about 1-8 J/cm²; or 1-7 J/cm², as mentioned above.

Suitably, the laser light may input an energy pulse to the skin in the range of about 1-50 mJ. In some embodiments, each pulse may have an energy in the range of about 1-30 mJ. For example, the pulse energy may be about 2.5 mJ, 5 mJ, 10 mJ, 15 mJ or 20 mJ.

In principle, it might be thought that smaller and smaller pulse energies would yield very fast heat diffusion times. In fact, there is a very strong lower cap on pulse energy. Given a pulse energy and required fluence for effective treatment, pulse effective area (spot size) may be calculated as: Area= pulse energy/ required fluence

It is well known that in biological tissue, pronounced scattering is caused by irregularities in refractive index by different cells, organelle and also macro features such as blood vessels and tissue types, etc. This effect is analogous to thermal diffusion as it can be approximated to a diffusion effect on the laser beam. Laser radiation, hitting the skin’s surface as a small round, uniform spot, for example, may start to diffuse sideways/radially and become a Gaussian-like shape as it propagates deeper into the skin. The greater the depth of penetration, the larger the width of the diffusion and the lower the average fluence. For a given depth and an infinitely small (i.e. narrow) beam, this effect creates a Gaussian “smear” of a typical length scale. As a finite input beam size gets closer to this length scale, the input fluence at the top of the tissue starts to decrease considerably while propagating downward. This limits the minimal useful spot size and thereby the fastest attainable thermal relaxation time of a single pulse. Suitably, therefore, the spot may have a diameter or other minimum dimension of at least about 0.1 mm; typically at least about 0.25 mm.

By using a small spot size in accordance with the present invention to allow the temperature of the skin to fall more quickly after irradiation with a pulse of laser light, the level of pain and extent of skin damage experienced by a subject may be reduced significantly. Not only does this make the process of pigment removal more tolerable for the subject, but it may also allow more frequent treatments, thereby accelerating the time taken for complete removal of a tattoo or other unwanted pigmentation. For instance, while prior laser methods of pigment removal require rest periods between successive treatments of about 6-8 weeks, the methods of the present invention may allow treatment to be safely repeated every 1-2 weeks; sometimes less. This represents a significant acceleration of the pigment removal process.

According to a fourth aspect of the present invention therefore, there is provided a method of pigment removal comprising a plurality of successive dermatological treatments in which a pulsed beam of laser light is moved over an area of a subject’s skin to be treated such that each pulse impinges on a different portion of the subject’s skin within the area to be treated; wherein each pulse has a fluence in the range of about 0.5-10 J/cm² and impinges on the subject’s skin in the form of a spot that is sufficiently small that the skin is not subjected to an elevated temperature greater than 44° C. for longer than the threshold duration at which damage to the skin results at the elevated temperature; wherein the dermatological treatments are repeated every 1-3 weeks.

Within each treatment, the area of the subject’s skin may be irradiated such that each separate portion of the area receives just one pulse of the laser light. In some embodiments, several treatments may be carried out on the same day, followed by a rest period of 1-3 weeks. Suitably, a maximum of four treatments may be carried out on the same day; more typically 1-3 treatments. In some embodiments, two treatments may be performed on the same day.

Suitably, the rest period may be 1-2 weeks. In some embodiments, the rest period for a particular subject may be determined by dermoscopy. In particular, changes to the subject’s skin may be observed after a treatment to determine when the subject is ready to receive a further treatment. Changes to the skin that may be monitored include, for example, scarification, skin damage and/or dermal blood vessel activity. Such techniques are well known to skilled dermatologists.

In some embodiments, the intensity of the light may be selected accordance with the present invention to interact with multiple colours within the visible spectrum. Thus, the laser light may have an intensity in the range 10¹¹-10¹² W/cm² as described above. However, according to the present aspect of the invention, a lower intensity of laser light may be used for interacting with a single colour. In some embodiments, therefore, the intensity of the laser light may be in the range 10⁹-10¹⁰ W/cm².

The fluence of each pulse in skin depth may be in the range of about 1-8 J/cm²; or 1-7 J/cm².

In some embodiments, laser light of two or more different wavelengths may be used in combination for increased efficacy against certain colours of pigment or ink. Thus, in some embodiments, a first pulsed beam of higher intensity laser light may be used in combination with a second pulsed beam of lower intensity laser light. The first beam may have an intensity of 10¹¹-10¹² W/cm² while the second beam may have an intensity of 10⁹-10¹⁰ W/cm². As described above the beams may have the same or similar fluences. For example, each of the first and second pulsed beams may independently have a fluence in the range 0.5-10 J/cm², as described above. It has been found, for example, that for some subjects it can be advantageous to target red pigment separately from other colours. Accordingly, in one embodiment a high intensity infra-red (IR) laser may be used in combination with a low intensity green laser. In another embodiment, two IR lasers may be used; one of relatively high intensity and one or relatively low intensity. Suitably, the high intensity IR laser may have a wavelength of 800 nm or 1030 nm. The relatively low intensity IR laser may have a wavelength of 1064 nm. The green laser may have a wavelength of 532 nm. In accordance with the invention, successive pulses of laser light may be applied to separate portions of the area as described above. The beam may be moved progressively over the area to be treated such that each pulse falls on a different portion of the subject’s skin. It will be appreciated that if the spots were to fill completely the area to be treated, and if the pulse repetition rate were so high that the whole area would be covered within a time that were much shorter than the thermal relaxation time for a single pulse, then it would, in effect, just create a single large spot and heat diffusion would be similar to that for a large, high energy spot. However, in some embodiments, the present invention comprehends incorporating deliberate, designed or controlled spacing between laser spots.

According to the present invention, therefore, the separate portions of the area of skin that are treated with successive pulses of laser light may advantageously be separated from each other by a small distance. Suitably, there may be a controlled spacing between the laser spots. In some embodiments, for example, the separate portions may be separated by at least about 0.1 mm.

In a fifth aspect of the present invention therefore, there is provided a method of dermatological treatment comprising moving a pulsed beam of laser light over an area of a subject’s skin to be treated; wherein the beam forms a spot of laser light on the subject’s skin and successive pulses fall on different respective portions of the area with a fluence in skin depth in the range of about 0.5-10 J/cm², and the portions are separated from one another by at least about 0.1 mm.

The separate portions may thus correspond to an array of small spots. An advantage of using such an array of small spots that are slightly distant from one another is that there will be small untreated patches of skin between neighbouring spots. As well as accelerating thermal relaxation and reducing damage, these small patches may be instrumental in substantially speeding up recovery processes of the skin that has been treated and suffered local damage. Whilst the patches between neighbouring spots are untreated within a single treatment, it will be understood that across a series of treatments, the spots within the area to be treated will not be in exactly the same place for each treatment and patches that are untreated in one treatment will be treated in another treatment. Fast healing makes this a favourable trade off in terms of overall treatment time.

Another option in accordance with the invention is to depart from a uniform circular radiation profile of the incoming laser and to introduce a gradient, such for example as a Gaussian profile. This may ensure that at the edges of the beam, where radiation is substantially lower, reduced or no damage will occur, even if adjacent spots cover the entire area of treatment. Suitably, the beam of laser light may be attenuated in an outer peripheral region such that the intensity of the beam within each spot is feathered in the outer peripheral region. For a circular spot, the outer peripheral region may be annular. The intensity within the outer peripheral region may be uniform such that there is a step change in intensity between the outer peripheral region and the rest of the beam. Alternatively, the intensity within the outer peripheral region may be graduated such that the intensity becomes progressively lower towards the outside of the beam.

Thus, in a sixth aspect of the present invention there is provided a method of dermatological treatment comprising irradiating an area of a subject’s skin to be treated with a pulsed beam of laser light in which each pulse produces a spot of laser light in the subject’s skin and has a fluence in the range of about 0.5-10 J/cm²; wherein the beam of laser light is moved over the area to be treated such that successive pulses fall on different portions of the area, and the beam is attenuated such that its intensity is lower in a peripheral outer region than the rest of the beam.

In some embodiments, therefore, the spots of laser light may overlap one another. In some embodiments, the laser spots may cover the entire area to be treated.

Yet another option in accordance with the invention for allowing heat to dissipate as quickly as possible from each portion of the area to be treated that is irradiated with a pulse of laser light is to ensure that immediately succeeding pulses have enough temporal delay between them to allow heat to diffuse from the portion treated. In some embodiments, this may be achieved by using a suitably low pulse repetition rate. In this respect, a pulse repetition rate in the range of about 1-10 Hz may be employed.

In all of these options, the fluence of each pulse in skin depth may be in the range of about 1-8 J/cm²; or 1-7 J/cm².

In some embodiments, the pulses of laser light may be directed onto the skin in a pattern comprising multiple adjacent rows of spots that are irradiated according to a sequence which ensures that neighbouring rows are not irradiated consecutively. For example, a series of consecutive pulses may be directed onto the skin in one line (i.e. as a row of spots) and, immediately thereafter, a further series of pulses may be directed onto the skin in another line that is spaced from the one line such that none of the spots of the other line is adjacent any of the spots of the one line. This ensures that the heat from the spots of the one line has time to dissipate at least in a direction transverse the one line. In some embodiments, the area to be treated may be irradiated by moving the laser beam relative to the area to be treated in a sequence of progressive linear or curvilinear passes, each pass having a duration of multiple pulses of laser light which thus impinge on the skin to treat a line of adjacent portions of the area of skin to be treated. The lines of portions are adjacent one another so as to cover the area to be treated. According to the present invention, the sequence of consecutive passes of the laser may be such that adjacent lines are not treated consecutively. Instead, each consecutive pass may create a line of spots that is non-adjacent to the immediately previous line of spots; a line of spots that is adjacent to a previous line may be made only after making one or more non-adjacent lines in the interim.

Suitably therefore the beam may be scanned over the area to be treated. Laser scanners are readily available with large scanning fields of more than 100x100 mm² for a 160 mm focal length. The selection of scanner focal length and size is a tradeoff of required scanning field area versus size (weight). A scanning mechanism may be advantageous for use with smaller spot sizes of the kind described above and higher pulse repetition rates as described in more detail below. This may allow for uniform application of laser spots and fast coverage. Further, accuracy of placement may be achieved by automated scanning of the laser beam on the subject’s skin. By using a small laser spot, small features on the subject’s skin may be treated accurately. Any suitable laser scanning system known to those skilled in the art may be used for this purpose. Embodiments of suitable beam scanning devices are described in more detail below.

In some embodiments, the radiation may be delivered through an articulating arm to the subject’s skin. A free end of the articulating arm may be held by a robot, an x-y or x-y-z stage or any other suitable means with sufficient degrees of freedom to scan precisely the laser beam in synchronization with a pulse rate of the laser and a required size of field. A part of the subject’s body where the area to be treated is located may be held stationary by any suitable apparatus.

In some embodiments, the laser beam itself may be scanned over the area to be treated by means of optical beam steering. Any suitable method of beam steering known to those skilled in the art may be used such, for example, as using galvanometric mirrors or acousto-optic modulators.

An advantage of this aspect of the present invention is that the beam of laser light may be scanned over a configurable area of the subject’s skin. The scanned beam may define a scanning field. In some embodiments, the scanning field may have a fixed shape, e.g. rectangular or circular. In some embodiments, the shape of the scanning field may be adjustable. For instance, the shape of the scanning field may be selectable from a number of pre-set shapes, e.g. circular, square and rectangular. A number of different rectangular field shapes may be provided, for example having different aspect ratios. In some embodiments, as described in more detail below, the shape of the scanning field may be configurable automatically according to the shape of the area to be treated. The scanning field may have a longest dimension of up to about 10 mm or about 12 mm Suitably, the scanning field may have a longest dimension of up to about 5 mm. In some embodiments, the field size may be adjustable. The field size may be continuously adjustable, or may be selected from a number of pre-set field sizes, e.g. 1, 2, 3 4 and 5 mm.

Suitably, the laser beam may be delivered via a work head. In some embodiments, the scanning field may cover the whole area to be treated. However, for treating areas that are larger than the size of the scanning field, the work head may be movable relative to the area to be treated. For example, in some embodiments, the work head may be carried on an end of an articulating arm of the kind described above. The scanner head may be movable manually. In some embodiments, as described in more detail below, the scanner head may be moved automatically. Alternatively, the scanner head may be stationary and the area to be treated may be moved in a controlled way relative to the scanner head. For example, the subject may be moved relative to the work head by means of a stage. Suitably, the stage may be automated, for example using means similar to those for the articulating arm. In this case, the subject, or at least a part of the subject’s body where the area to be treated is located, may be supported immovably on a support member such, for example, as a movable stage, that is arranged for controlled movement relative to the work head.

Advantageously, the work head may include a spacer of fixed or adjustable length that extends way from the work head in a direction substantially parallel to the direction of the laser beam in use. The spacer may suitably comprise an elongate member that extends juxtaposed the treatment laser beam without obstructing it. For instance, the elongate member may comprise a finger-like member or a tubular or semi-tubular member that surround the laser beam. The elongate member may have a smooth distal end remote from the work head for contacting the subject’s skin. The spacer thus serves to maintain the work head at a constant distance from the area of skin to be treated. Suitably, the spacer may be detachable from the work head. The spacer member may be disposable or washable so that a clean, sterile spacer may be used for each subject to be treated.

A further advantage of scanning multiple small spots in accordance with the present invention is that the overall shape of the scanned area can be controlled for optimising treatment. In previous methods, a single, large spot is typically round or rectangular, and while its overall size can be controlled, its shape cannot be. By contrast, in some embodiments of the present invention, the scanning area or scanning field may be programmed to any desired shape. The scanning field may have an selectable “brush” size and/or shape which can be adapted according to the shape of the area to the treated. This may be beneficial, for example, for scanning elongated shapes to remove long thin tattoo lines, e.g. text characters that typically have an abundance of horizontal and vertical lines. Within the scanning field, a corresponding part of the area of the subject’s skin is irradiated rapidly once. If necessary, the scanning head may then be moved to another part of the area to be treated and so on as described above until the entire area has been covered. A throughput advantage as compared to using just a small round or square spot may be considerable.

The present invention thus comprehends the use of a scanning pattern of arbitrary (brush) shape for optimisation of treatment efficacy and minimisation of treatment duration by considering the shape of the target area for treatment. A further advantage of a scanning field of adjustable size and shape is that it may minimise lasing of un-tattooed or unpigmented skin to reduce collateral tissue damage and so reduce healing time.

Suitably, a visible aiming beam may be directed continually around the periphery of the scanning field defined by the treatment beam, for example by scanning, to show the outline of the scanning field, thereby to assist an operator in guiding the laser beam over the area to be treated in the manner described above. It will be understood that the aiming beam should have no effect on the subject’s skin but serves only to provide a visible indication on the skin of the position of the treatment beam. Once the operator is satisfied that the scanning field is correctly located relative to the subject’s skin they may selectively operate the pulsed laser beam to scan across the field.

Those skilled in the art will appreciate that any combination of the above-mentioned techniques for scanning the laser beam over the area to be treated may be employed.

In some embodiments, a smart scanning technique may be employed to ensure maximal delay between irradiation of neighbouring portions of the subject’s skin, thereby to reduce thermal load. Thus, the beam may be scanned over the scanning field or area to be treated in a configurable scanning pattern that reduces thermal load by skipping a portion of the area to be scanned adjacent to a portion just scanned and returning to it after scanning portions more distant from scanned portion. Suitably, the beam may be scanned across the area to be treated as a series of linear or curvilinear lines.

Thus, in an seventh aspect of the present invention there is provided a method of dermatological treatment comprising irradiating an area of a subject’s skin to be treated with a pulsed beam of laser light in which each pulse produces a spot of laser light in the subject’s skin and has a fluence in the range of about 0.5-10 J/cm²; wherein the beam of laser light is scanned over the area to be treated in a series of linear or curvilinear lines such that successive pulses fall on different portions of the area to be treated.

In some embodiments, the beam may be scanned across a scanning field of the kind described above. To cover the area to be treated, or the scanning field, the lines may be scanned in juxtaposition with one another. Each line may comprise a plurality of successive pulses of the laser light to adjacent portions of the subject’s skin, which may be overlapping or non-overlapping as described above. Suitably, adjacent lines may be scanned in succession. In some embodiments, the beam may be raster scanned over the area to be treated. Alternatively, the lines may be interlaced. In another embodiment the portions within each scanned line may be irradiated out of order. Thus, each line may be scanned in a sequence of passes in which selected, non-adjacent portions are irradiated. For example, within each line, every n^{th} portion may be irradiated starting with a first portion up to the end of the line, and then every n^{th} portion starting with a second portion which was not irradiated in the first run and so on until all portions are irradiated during the compete sequence. In some embodiments, the beam may be scanned over the scanning field in a pattern which comprises a plurality of adjacent rows, each row comprising a line of adjacent portions of the subject’s skin to be irradiated by respective pulses of the laser. All of the rows may be scanned a plurality (n) of times and all of the rows may be scanned before any row is repeated. Within each scan of a given row, only every n^{th} portion may be irradiated, and the every n^{th} portions in adjacent rows may be offset so that the every n^{th} portions in each row may be non-adjacent the n^{th} portions in the adjacent rows. Each time the rows are scanned, different n^{th} portions are irradiated that were not previously irradiated. After n scans of the rows, all of the portions may be irradiated, thereby covering the whole scanning field. For instance, each row may be scanned twice and within each row every second portion may be irradiated with the laser, the every second portion in each row being offset with respect to, and thus non-adjacent to, the every second portions in the adjacent row(s), whereby the scanning field may be scanned in a “checkers board” type pattern The scanning pattern may be circular/annular, helical, quadrilateral, consist of concentric rings, or any other shape to optimise the treatment process.

It will be appreciated that the required scanning time for multiple small portions of the subject’s skin will be cumulatively longer than the scanning time for a single larger spot according to previous methods. However, the increased scanning time occasioned by the use of a small (sub-millimetre) spot size in accordance with the present invention may be ameliorated by increasing the pulse repetition rate of the beam of laser light. Suitably, therefore, the pulsed beam may have a pulse repetition rate of more than about 30 Hz. In some embodiments, the pulsed beam may have a pulse repetition rate of more than about 100 Hz. In some embodiments, the pulse repetition rate may be up to 1 KHz or more. For example, in some embodiments a laser having a pulse repetition rate of 2000 Hz, 4000 Hz or even 6000 Hz may be used. Given the inevitable inclusion of other steps in treating a subject that increase the overall time required for the treatment (e.g. placement of the laser by the operator) a pulse repetition rate in the range of about 200-500 Hz has been found be sufficient in most cases to ensure no material loss of throughput as compared with prior methods.

As described above, the present invention thus comprehends positioning a scanning head relative to an area of a subject’s skin to be treated for delivering a pulsed beam of laser light to the area to be treated in accordance with one or more aspects of the invention as described above. The laser beam may be scanned automatically across a scanning field of adjustable size and/or shape, and an outline of the scanning field may be shown to an operator for ensuring correct location of the field on the subject’s skin using an aiming beam. Upon firing the laser, each portion of the subject’s skin within the field is irradiated with just one pulse of laser light. It will be understood that for a square scanning field having a size of, say, 5 mm and a spot size of 0.1 mm, approximately 3000 pulses will be required to scan the beam across the whole field. For a larger spot size of say 1 mm, only about 30 pulses will be needed. In general, the number of pulses of the laser required to irradiate the entire scanning field may range from 1 to about 10,000, more typically about 100 to about 1000. At a pulse repetition rate of 100 Hz to 1 KHz, the time required to scan the whole field may be about 10 ms to about 100 s, more typically about 100 ms to about 10 s. After the scanning field has been scanned, the scanning head may be repositioned over a different part of the subject’s skin if required, i.e. where the area to be treated is larger than the field or where the area to be treated is contoured such that it cannot be treated in its entirety with the scanning head in one position.

In some embodiments, the shape of the scanning field may be calculated automatically by acquiring the shape of the area to be treated using optical or other means. For example, the shape of the area to be treated may be determined by camera and/or machine vision. An optical trace may be used to display the calculated shape of the scanning field in relation to the subject’s skin to an operator for verification prior to operation of the laser. For instance, the optical trace may indicate an outline of the calculated scanning field. Alternatively, the calculated field shape and its position on the subject’s skin may be displayed to the operator on a suitable screen.

The scanning head may therefore include at least one camera for acquiring images of the subject’s skin to be processed to determine the area to be treated and the corresponding required scanning field size and shape. The images may be processed using a suitable image recognition system. The scanning head may further comprise one or more lamps for illuminating the subject’s skin to ensure the acquisition of images of usable quality.

In accordance with an eighth aspect of the present invention therefore there is provided a method of dermatological treatment which comprises using a camera to acquire one or more images of at least part of an area of a subject’s skin to be treated; processing the one or more images using an image-recognition technique to determine the shape and size of the at least part of the area to be treated; adjusting a shape and size of a scanning field for a pulsed laser beam according to the determined shape and size of the at least part of the area to be treated; and thereafter scanning the pulsed beam of laser light onto the at least part of the area to be treated over the whole of the scanning field.

The methods of the present invention may therefore comprise a sequence of method steps, which may be performed under the control of any suitable computing system. Each of the method steps may represent an algorithm whose structure may include and/or may be represented by multiple sub-steps.

In accordance with a ninth aspect of the present invention there is provided laser treatment apparatus for dermatological treatment comprising; a work head that includes a beam scanner for scanning a treatment laser beam with a spot size of less than 2 mm over a scanning field of adjustable size and/or shape and a camera; an optical input for connecting the beam scanner to at least one pulsed treatment laser; an adjustable positioning device for stably positioning the work head adjacent an area of a subject’s skin to be treated and an automatic control system for controlling operation of the laser treatment apparatus; wherein the automatic control system is configured to receive one or more images of the area to be treated from the camera, process the received images to determine the shape of at least part of the area to be treated, adjust the size and/or shape of the scanning field according to the determined shape of the at least part of the area to be treated and to scan the treatment laser beam over the scanning field.

Suitably, the automatic control system may comprise a computer and software, which, when executed by the computer, causes the laser treatment apparatus to operate as described herein. Since computers and software are well known to those skilled in the art, it is unnecessary to describe in detail herein how the invention should be implemented using such equipment. In some embodiments, however, it will be understood that automatic control system may comprise at least one physical processor and physical memory comprising computer-executable instructions which, when executed by the physical processor, cause the physical processor to carry out at least one method in accordance with the invention.

Suitably, image recognition of the at least part of the area to be treated may be carried out using standard methods known in the art of image analysis. Multi-spectral imaging may be used to provide additional information for finding the right target shape of a lesion, for example, that should be treated. Additionally, machine-learning and/or artificial intelligence methods may be used for recognising the area to be treated.

As mentioned above, the work head may further comprise one or more lamps for illuminating the area to be treated to ensure the images captured by the camera are of sufficiently good quality to facilitate their processing and/or image recognition. Suitably, for example, the work head may include one or more LEDs that are operable to emit light in the visible range, e.g. white light, to illuminate the area of the subject’s skin to be treated. Such light may assist image recognition of the at least part of the area to be treated.

The work head may further comprise a optical tracer for indicating an outline of the scanning field to an operator on the subject’s skin. The automatic control system may be further configured to control the optical trace device for displaying an outline of the scanning field, adjusted according to the determined shape and size of the at least part of the area to be treated, on the subject’s skin.

In some embodiments, the laser apparatus may further include a display that is adapted to receive a display signal from the control system representing images of the at least part of the area of the subject’s skin to be treated and to display those images on the screen. The automatic control system may be further configured to display on the screen an outline of the scanning field, adjusted according to the determined shape and/or size of the at least part of the area to be treated, superposed on the images of the subject’s skin.

Suitably, the automatic control system may be configured to wait for receipt of a safety control signal from the operator after the scanning field has been indicated before operating the beam scanner. The safety control signal may be generated by a suitable trigger device that is selectively operable by the operator; for example, a foot pedal, a switch, a button or the like. In some embodiments, the control system may be configured to allow the operator to adjust the shape and/or size of the scanning field via a suitable input device such, for example, as a keyboard, touch-screen, selection buttons, a rotatable knob or dial or the like or a combination of two or more of these.

In some embodiments, the work head may further comprise an aiming beam device for emitting a visible aiming beam towards the subject’s skin for indicating on the subject’s skin the position of the laser beam scanner relative to the area to be treated, thereby to facilitate adjustment of the positioning device to position the work head correctly adjacent the area of the subject’s skin to be treated. Conveniently, the aiming beam may be generated by an optical trace device of the kind mentioned above, which may thus be configured for selectively generating an aiming beam for locating the work head and an optical trace for verifying the shape and/or size of the scanning field calculated by the automatic control system, optionally adjusted by the operator, before firing the treatment laser.

A further complication is that, in general, a subject’s skin is not flat. This implies that there will be a limit to the area of the skin that can be scanned with the treatment laser beam, even if the area to be treated is smaller than the maximum available scanning field of the beam scanner. The angle of incidence of the laser beam onto the target area may further limit the attainable area. In some embodiments, the topography of the area to be treated may be addressed by adjusting the focal length of the beam scanner, but this too might have limits to attainable correction. By way of example, consider a port wine stain lesion or other area to be treated that extends around a subject’s wrist; a scanner would be able to cover only part of the wrist per scan.

To address this problem, in some embodiments, the laser treatment apparatus of the present invention may be adapted to determine the topography of the at least part of the area to be treated and to calculate an attainable scanning field owing to height changes, angle of incidence and optionally other limitations. In some embodiments, the laser treatment apparatus of the invention may therefore further comprise one or more topograhy measuring instruments for measuring the topography of the at least part of the area to be treated. For example, the laser treatment apparatus may include a distance measuring device for measuring the distance to various parts of the area to be treated. Suitably, the distance measuring device may be built into the work head. Suitable distance measuring devices are known and available to those skilled in the art, including, for example, 3D cameras, dedicated measuring systems and 3D scanners. In some embodiments, an aiming beam of the kind described above may be used to perform triangulation for calculating the topography. The automatic control system may be further configured to determine the topography of the at least part of the area to be treated based on measurements of this kind. The control system may be configured to fuse the shape and size and the topography of the at least part of the area to be treated in order to calculate the shape and/or size of the attainable scanning field.

In some embodiments, the contour limitation of the target lesion or other area to be treated may be such that it extends beyond the attainable scanning field of a single scan. In some embodiments, the lesion or other area to be treated may be too large for a single scan, even for a flat topography. In accordance with the present invention, the positioning device may allow the work head to be positioned in multiple different locations to allow treatment of the whole area to be treated in multiple segments. Thus, the work head may be positioned in a new location that covers a different part of the subject’s skin for complete coverage of the required area to be treated.

In some embodiments, the work head may be repositioned manually by the operator. At each position, the control system may be operable to process one or more images of an adjacent segment of the area of the subject’s skin to be treated as described above to detect the boundaries of the segment. Suitably, the control system may use an image processing method that relies on images of untreated skin to identify a segment of the area to be treated. The requisite shape and size of the scanning field for treating the segment may then be determined as described above. The treatment laser may then be operated to irradiate the segment across the scanning field. Using an aiming beam of the kind described above, the operator may position the work head over each segment to be treated in turn such that it overlaps with one or more previously treated segments, usually including an immediately previously treated segment, if any. The operator may, for example, use “frosting”, which occurs as a result of laser treatment of skin, to identify previously treated segments. The automatic control system may be configured to mask parts of each segment that overlap previously identified segments at other positions of the work head, so that portions of the skin in areas of overlap between two or more adjacent segments are not irradiated more than once within a single treatment. An image stitching algorithm of the kind known to those skilled in the art may suitably be used to identify areas of overlap between identified segments. At each position, the camera may have a field of view that is larger than the attainable scanning field to facilitate identification of segments of the area to be treated using untreated skin.

Alternatively, the positioning device may be automated and the automatic control system may be further configured for controlling the positioning device to position the work head. In this way, the work head may be positioned automatically to scan successive scanning fields. The successive scanning fields may be contiguous to one another to ensure complete coverage of the area to be treated. Alternatively the successive scanning fields may overlap and an image stitching algorithm of the kind described in the preceding paragraph may be used to mask areas of overlap. An example of a suitable automated positioning device is a robotic arm with sufficient degrees of freedom for covering the entire area to be treated. The robotic arm may be capable of monitoring and recording its position as described below.

Suitably, the automated positioning device may be switchable between a first mode in which the work head can be moved freely by the operator and a second mode in which the position of the work head can only be adjusted under the control of the control system. The positioning device may comprise, for example, at least one selectively operable clutch to allow the positioning device to be selectively switched between these two modes. In the first mode, the positioning device may be moved by the operator without resistance but holds the work head sufficiently stably that it does not move, for example under gravity, if it is released by the operator.

In the first mode, the work head may be manipulated by the operator to capture one or more images the whole of the area to be treated. In the second mode the work head may be moved automatically over the area to be treated under the control of the automatic control systemfor treating the subject’s skin with the pulsed laser light in accordance with the invention.

In some embodiments, the automatic control system may be configured to operate a learning mode and a scanning mode. In the learning mode, the operator holds the work head with the positioning device (e.g. a robotic arm) in the first mode and manipulates it around the whole of the area to be treated, across the entire contour. The work head may follow the guidance of the operator without resistance in the first mode. The camera is operated continuously to capture images of the subject’s skin while the robotic arm continuously measures its position on all axes and records its motion and the path followed by the operator. At the end of this sequence, the automatic control system has received multiple data-sets: the captured images, the positions and trajectory of the work head and 3D contour and distance measurements of the area to be treated. The automatic control system is configured then to calculate a scanning path by optimizing the scan path. As a first approximation, the path taken by the operator in the learning sequence may be used.

Alternatively, an independent 3D scanner may be used to scan the subject, and the operator may input the area to be scanned on a computer. Based on that definition, the control system may then calculate a required 3D trajectory of the scanner head.

In the scanning mode, the positioning device is switched to the second mode, and the work head is moved under the control of the control system to follow the path generated by the control system while operating the beam scanner in successive scanning fields to scan the pulsed laser beam over the whole of the area to be treated.

In some embodiments, the laser treatment apparatus may further comprise an interlock device to be operated by the subject receiving treatment. The automatic control system may be configured such that it can only be operated when the subject is positively operating the interlock device. The interlock device may comprise any suitable button, trigger, switch or the like that the subject can hold in their hands during treatment. Suitably, the interlock device is non-latching. If the subject releases the interlock device during operation of the laser treatment apparatus, an interlock control signal is sent to the control system causing the laser apparatus immediately to pause its operation. Thereafter, if the subject re-operates the interlock device, the control system may be configured to wait for the operator to provide again the requisite input signal by operation of the trigger device mentioned above. In this way, if the subject feels anxious about movement of the robotic arm or any other positioning device during treatment, they can cause the apparatus to stop. Where the laser beam is scanned mechanically over the area to be treated by a robotic arm, x-y or x-y-z stage or the like, it will be appreciated that the movement may be very fast and intimidating. However, where an optical scanner is used, the fast motion is only optical and the positioning device is either stationary or only very slowly moving.

As treatment may take several minutes or more, it is inevitable that the subject will move to some degree. In some embodiments therefore, the laser treatment apparatus may further comprise one or more movement detectors for detecting movement of the subject; particularly movement of the area to be treated. The movement detectors may comprise a camera on the work head or a device for taking 3D measurements of the subject’s skin. The movement detector may be configured to detect movement of the subject, and the control system may be configured to correct automatically scanning of the treatment laser beam to compensate accordingly or to stop scanning, for example if a detected movement is too large or too fast for safe operation. Markers or indicators may be attached, adhered or drawn on the subject’s skin in some embodiments to facilitate measurement of motion of the subject.

The laser treatment apparatus of the invention is adapted for use with a pulsed laser. Suitably, therefore, the laser treatment apparatus may further comprise a pulsed treatment laser and an optical system for connecting the treatment laser to the optical input of the work head. In some embodiments, the laser may be a mode-locked laser capable of producing pulses of laser light with a pulse width of the order of picoseconds. As described above, in some embodiments, the pulses may have a pulse width in the range of about 0.1-100 ps and an intensity of at least about 50 GW/cm².

It will be understood by those skilled in the art that features of the invention that are described above in relation to the first to eighth aspects of the invention apply equally to the laser treatment apparatus of the invention and for conciseness need not be repeated here.

In accordance with a tenth aspect of the present invention therefore there is provided laser apparatus for dermatological treatment comprising a pulsed treatment laser, a work head for delivering a pulsed beam of laser light onto an area of a subject’s skin to be treated, and an optical system for connecting the treatment laser to the work-head; the arrangement being such that the pulsed laser beam has a pulse width in the range of about 0.1-100 ps and an intensity of at least about 50 GW/cm².

As described above, the fluence of each pulse in skin depth may be in the range of about 0,5-10 J/cm²^{,}; preferably about 1-8 J/cm² or about 1-7 J/cm².

In some embodiments, as described above, the beam scanner may be configured such that in use each pulse is delivered to the subject’s skin in the form of a spot having a maximum dimension in the range of about 0.1 to less than 2.0 mm; preferably about 0.5-1.0 mm.

According to an eleventh aspect of the present invention therefore there is provided laser treatment apparatus for dermatological treatment comprising a pulsed treatment laser, a work-head which includes a beam scanner for scanning a pulsed beam of laser light onto an area of a subject’s skin to be treated such that each pulse impinges on a different portion of the subject’s skin, and an optical system for connecting the treatment laser to the beam scanner; the arrangement being such that in use each pulse is delivered by the beam scanner into the subject’s skin in the form of a spot having a maximum dimension in the range of about 0.1 to less than 2.0 mm; preferably about 0.5-1.0 mm, and the fluence of each pulse in skin depth is in the range of about 1-7 J/cm².

Suitably, as described above, the beam scanner may be configured such that the different portions of the subject’s skin are separated from one another by at least about 0.1 mm.

In accordance with a twelfth aspect of the invention therefore there is provided laser apparatus for dermatological treatment comprising a pulsed treatment laser, a work-head which includes a beam scanner for scanning a pulsed beam of laser light onto an area of a subject’s skin to be treated such that each pulse impinges on a different portion of the subject’s skin, and an optical system for connecting the treatment laser to the beam scanner; the arrangement being such that in use each pulse is delivered by the beam scanner into the subject’s skin in the form of a spot with a fluence in skin depth in the range of about 1-7 J/cm² and the different portions are separated from one another by at least about 0.1 mm.

In some embodiments, as described above, the spots may be overlap with one another. The beam of laser light may be attenuated such that its intensity is lower in a peripheral outer region than the rest of the beam.

In a thirteenth aspect of the invention therefore there is provided laser apparatus for dermatological treatment comprising a pulsed treatment laser, a work-head including a beam scanner for scanning a pulsed beam of laser light onto an area of a subject’s skin to be treated such that each pulse impinges on a different portion of the subject’s skin, and an optical system for connecting the treatment laser to the beam scanner; the arrangement being such that in use each pulse is delivered by the beam scanner into the subject’s skin in the form of a spot with a fluence in skin depth in the range of about 1-7 J/cm² and the beam is attenuated such that its intensity is lower in a peripheral outer region than the rest of the beam.

Advantageously, the beam scanner may be configured, as described above, such that the beam of laser light is scanned over a scanning field in a series of linear or curvilinear lines such that successive pulses fall on different portions of the area to be treated.

In a fourteenth aspect of the invention there is provided laser apparatus for dermatological treatment comprising a pulsed treatment laser, a work-head including a beam scanner for scanning a pulsed beam of laser light onto an area of a subject’s skin to be treated, and an optical system for connecting the treatment laser to the beam scanner; the arrangement being such that in use each pulse is delivered by the beam scanning device into the subject’s skin in the form of a spot with a fluence in skin depth in the range of about 1-7 J/cm² and the beam scanner is configured such that the beam of laser light is scanned over a scanning field in a series of linear or curvilinear lines such that successive pulses fall on different portions of the area to be treated.

As described above, the pulsed beam may have a pulse repetition rate of more than about 30 Hz, preferably more than about 100 Hz; optionally 200-500 Hz. In some embodiments, the pulsed beam may have a pulse repetition rate of up to about 1000 Hz. Suitable pulsed lasers capable of the above-described requirement include the high pulse energy and high repetition rate picosecond laser that is commercially available from Photonics Industries of Long Island, NY, under the name RGL-1064-4 which is capable of delivering 4 mJ energy at 1000 Hz and the high energy KHz repetition rate picosecond amplifier available from Ekspla of Vilnius, Lithuania, under the name APL2201 which is capable of delivering 10 mJ at 1000 Hz.

Those skilled in the art will recognise that pulsed lasers of the kind mentioned above are very expensive pieces of equipment. It will also be appreciated that when treating a series of subjects, the laser is not in use for a substantial amount of time, which is typically about 50% of the time. As explained above, in some embodiments, the present invention utilises a fast pulse repetition rate to compensate for a smaller spot size as compared with previous treatment methods. In addition, pigment removal in accordance with the invention requires a minimum amount of energy per pulse; typically 1-50 mJ as mentioned above.

In some embodiments, a single treatment laser may be arranged to deliver pulsed laser light selectively to a plurality of laser treatment apparatus in accordance with the invention. Each laser treatment apparatus may be provided in a different treatment area such, for example, as a different treatment room. As described above, each of the laser treatment apparatus may comprise a work head and an optical input for coupling the work head to the treatment laser. An optical system comprising an opto-mechanical selector may be provided for selectively coupling the treatment laser to one of the laser treatment apparatus. In this way, the treatment laser can be used to deliver pulsed laser light to one of the laser treatment apparatus for treating a subject while the other laser treatment apparatus are not in use. Such an arrangement may allow for greater utilisation of the treatment laser.

In accordance with a fifteenth aspect of the present invention therefore, there is provided a dermatological laser treatment facility comprising a pulsed treatment laser; a plurality of separate treatment areas; a laser treatment apparatus in each treatment area, each laser treatment apparatus comprising a work head that includes a beam scanner for scanning a treatment laser beam with a spot size of less than 2 mm over an area of a subject’s skin to be treated and an optical input; and an optical system for connecting the treatment laser to the optical input of the work head of each laser treatment apparatus; wherein the optical system comprises an opto-mechanical selector that is operable for selectively steering the laser beam to any one of the laser treatment apparatus.

Suitably, the pulsed treatment laser may be operable to produce a beam of laser light having a pulse repetition rate of at least 30 Hz as described above.

Each pulse may have an energy of 1-50 mJ, preferably about 1-30 mJ. In some embodiments, the pulsed treatment laser may have a pulse repetition rate of greater than 100 Hz, preferably at least 200 Hz and more preferably at least 500 Hz. In some embodiments, the pulsed treatment laser may have a pulse repetition rate of 1000 Hz or more.

The laser light may be modulated electronically at the treatment laser according to the specific requirements of each treatment area.

In some embodiments, a high pulse energy treatment laser may be used. Rather than toggling the beam sequentially between treatment areas as described above, a passive optical splitter may be used to divide the beam for use by a plurality of laser treatment apparatus of the present invention in parallel. In this way, the laser treatment apparatus in different treatment areas may be used simultaneously.

In accordance with a sixteenth aspect of the present invention therefore there is provided a dermatological laser treatment facility comprising a pulsed treatment laser that is operable to produce a beam of laser light having a pulse repetition rate of at least 30 Hz; a plurality of separate treatment areas; a laser treatment apparatus in each treatment area, each laser treatment apparatus comprising a work head that includes a beam scanner for scanning a treatment laser beam with a spot size of less than 2 mm over an area of a subject’s skin to be treated and an optical input; and an optical system for connecting the pulsed treatment laser to the optical input of the work head of each laser treatment apparatus; wherein the optical system comprises a passive optical splitter for splitting the beam and directing it to each of the laser treatment apparatus in parallel.

Suitably, the pulsed treatment laser may have a pulse energy of at least 5 mJ,. In some embodiments, the pulsed treatment laser may have a pulse energy of up to about 300 mJ. For example, in some embodiments, a pulse energy of 10 mJ, 15 mJ, 20 mJ or 30 mJ may be used. The treatment laser may be operated continuously at full power output. The beam scanner in each work head may comprise a fast optical modulator such, for example, as a pockel cell, a galvo mirror or the like for modulating the divided laser beam according to the specific requirements of each separate treatment area.

Typically, the beam may be divided between at least two laser treatment apparatus/treatment areas. Owing to the properties of the treatment laser in terms of pulse repetition rate and pulse energy, there is a limit on the number of times the beam can be split whilst retaining within each daughter beam sufficient pulse energy for dermatological pigment removal as described herein.

In another variant of the invention, therefore, a beam multiplexing technique may be employed to divide a single pulsed laser beam between multiple treatment areas. An optical system that is connected to a treatment laser having a fast pulse repetition rate may include a plurality of optical modulators which are arranged in series and are independently selectively operable to pick selected pulses of a pulsed laser beam and direct them to a respective laser treatment apparatus according to the invention.

In accordance with a seventeenth aspect of the present invention therefore there is provided a dermatological laser treatment facility comprising a pulsed treatment laser that is operable to produce a beam of laser light having a pulse repetition rate of at least 30 Hz; a plurality (n) of separate treatment areas; a laser treatment apparatus in each treatment area, each laser treatment apparatus comprising a work head that includes a beam scanner for scanning a treatment laser beam with a spot size of less than 2 mm over an area of a subject’s skin to be treated and an optical input; and an optical system for connecting the pulsed treatment laser to the optical input of each laser treatment apparatus; wherein the optical system comprises a plurality (n) which is equal to the number of treatment areas of optical modulators that are arranged in series and are selectively operable to steer successive pulses to different ones of the laser treatment apparatus in turn, with each optical modulator picking, every nth pulse.

Examples of optical modulators include pockels cells, fast galvo mirrors, rotating polygon scanners and others known to those skilled in the art.

In this way, a single pulsed treatment laser with a high pulse repetition rate may be used to supply pulses of an undivided beam of laser light to a plurality of different laser treatment apparatus by repeatedly selectively directing successive pulses to different ones of the laser treatment apparatus up to a number of successive pulses equal to the number of laser treatment apparatus. Thus, each treatment apparatus may selectively receive pulsed laser light at a frequency equal to the original frequency of the pulsed treatment laser divided by the number of treatment apparatus. For instance, if the laser is used to supply three treatment apparatus then the available pulse repetition rate for each treatment apparatus is equal to one-third of the pulse frequency of the treatment laser. For this reason, it is desirable to use as fast a treatment laser as possible, preferably with pulse repetition rates in excess of 100 Hz, preferably in excess of 200 Hz and more preferably greater than 500 Hz. In some embodiments, pulse repetition rates of 1000 Hz or more may be used. In some embodiments, pulse repetition rates of up to 2000 Hz, 4000 Hz or even 6000 Hz may be used in order to minimise treatment times for treating multiple subjects simultaneously using a single laser beam.

It will be understood that pulses of the laser beam are only directed to a given laser treatment apparatus when the associated optical modulator is actuated. If the optical modulator is not actuated, the pulses of energy continue within the optical system until they are received in a suitable beam dump provided for that purpose.

Alternatively, pulses may be continuously directed to different treatment areas, while an additional optical coupler may be added in series in each area to modulate the pulses that are required for treatment in each area.

As described above, each pulse should have an energy of about 1-100 mJ. Suitably the pulses received in each laser treatment apparatus may be further modulated by the respective beam scanner to a pulse duration that is calculated to provide a fluence at skin depth in the range of about 0.5-10 J/cm2. Suitable pulse durations, spot sizes and intensities are described above.

It will be appreciated that it is important to ensure that laser treatment apparatus for use on a person’s skin will operate reliably and consistently within required operating parameters as described above, including, for example, the power and/or position of the laser beam incident on the area of skin to be treated. To this end the laser treatment apparatus of the present invention may include test apparatus which incorporates one or more sensors for testing one or more physical and/or operating characteristics of the laser beam and/or work head.

Suitably, the test apparatus may comprise a supporting structure including a work head engaging portion that is configured to engage with the work head and at least one sensor that is fixed to the supporting structure at a position spaced from the work head engaging portion. Suitably, the sensor(s) may be fixed to the supporting structure such that when the work head is engaged with the work head engaging portion, the distance between the sensors and the work head is substantially the same as the distance between the work head and the skin when the laser treatment apparatus is in use. In this way, the sensor(s) may be located in a plane that is optically equivalent to the plane of the skin when the laser treatment apparatus is in use. As described above, the work head may be adapted for connection to a detachable spacer. The work head may therefore include a spacer engaging portion. The work head engaging portion of the supporting structure may be configured for releasable engagement with the spacer engaging portion of the work head.

Advantageously, the supporting structure may comprise a perforated separator plate interposed between the work head engaging portion and the at least one sensor. The separator plate may be formed with one or more holes that extend therethrough. The remainder of the separator plate may be opaque to laser light emitted by the work head. The one or more holes may be formed in the separator plate at known locations relative to the work head engaging portion. Thus the separator plate is suitably fixed to the supporting structure in an accurate position. The separator plate may therefore be used to detect a perturbation of a laser beam scanner in the work head. In a test mode, the beam scanner may be operated to steer the laser beam through the one or more holes to ensure the beam scanner has not wandered out of alignment and that the beam scanner is operating as expected. One more light sensors are provided on the supporting structure on an opposite side of the separator plate from the work head engaging portion to detect light passing correctly through the one or more holes.

In some embodiments, a position sensitive detector of the kind available to those skilled in the art that is capable of measuring the position and power of the laser beam may be used instead of or in addition to the perforated separator plate. In some embodiments, the separator plate may be formed with a plurality of holes of different sizes to account for divergence of the beam. The one or more sensors may include a corresponding number of power sensors for measuring the power of the laser beam emitted by the work head, each power sensor being associated with a respective one of the holes. It will be appreciated that holes of different sizes will result in different power levels to be measured by the respective power sensors.

Suitably, the supporting structure may support one or more lenses between the one or more sensors and the work head engaging portion for adapting the optical path of the laser beam to the test apparatus. At least one lens may be located between the separator plate and the work head engaging portion.

Conveniently, the supporting structure of the test apparatus may comprise a holder for removably holding the work head when it is not in use.

Following is a description by way of example only with reference to the accompanying drawings of embodiments of the present invention.

### BRIEF DESCRIPTION OF THE DRAWINGS

In the drawings:

FIG. 1 is a schematic side view of a dermatological laser treatment facility which includes laser treatment apparatus according to a first embodiment of the present invention.

FIG. 2 is a schematic section side view of an optical path and scanner head of the dermatological treatment apparatus of FIG. 1 which is shown connected to a pulsed laser via an articulating arm for laser beam delivery to a subject. The work head includes a galvanometric x-y scanner as a beam steering apparatus. The beam is then focused to reach a desired size on subject’s skin.

FIG. 3 is a side elevation of a manually movable scanner work head forming part of the dermatological laser treatment apparatus of FIG. 1.

FIG. 4 is a rear elevation of the work head of FIG. 3.

FIG. 5 shows various scanning fields of different sizes and shapes which are selectable in the work head of the system in FIG. 1

FIG. 6 is a flow chart of the operation of the dermatological laser treatment apparatus of FIG. 1

FIG. 7 is a flow diagram of a method for removing a tattoo or other pigmentation from a subject’s skin in accordance with the present invention.

FIG. 8A illustrates treatment of an area to be treated in accordance with a prior method. FIG. 8B illustrates shape/size optimisation of the scanning field in accordance with the present invention In the prior method as shown in FIG. 8A, an exemplary thin stem of a rose-shaped tattoo requires a very small spot size; a large number of spots with exact placement are needed to cover the entire stem. By contrast, in the scanning method of the present invention, the scanning field may be programmed to any desired shape. An elongated rectangular scanning field, for example, may be more efficient and faster for treating long, thin tattoo lines.

FIG. 9 is a flow chart of a method for determining a required working fluence (intensity) for pigment removal using pulsed laser light of different pulse widths.

FIG. 10 is a chart of measured ablation threshold fluence vs. pulse width for various colours of pigment. It is evident that shorter pulse widths require less fluence, and difference in threshold between different colours is smaller.

FIG. 11 shows an example of multicolour tattoo removal in accordance with the present invention using a high intensity laser as compared with a prior method using a low intensity laser. Depicted are photographs showing actual removal results in a controlled experiment performed on live porcine skin.

FIGS. 12A and 12B show skin temperature profiles over time. FIG. 12A shows the temperature profile resulting from an high energy large spot. FIG. 12B shows the temperature profile resulting from a low energy small spot. Onset of heat diffusion is noticeable at time scales of about 10 s for the large spot. For a small spot, diffusion of heat is noticeable after less than about 0.1 s.

FIG. 13 is a chart showing the temperature at the centre of a volume of a heated tissue as a function of time. The thermal relaxation time (50% of initial temperature delta) for a 1 mJ, 0.22 mm spot is about 0.12 s; for a 5 mJ, 0.5 mm spot, the thermal relaxation time is about 0.7 s; and the thermal relaxation time for a 500 mJ, 5 mm spot is about 70 s.

FIG. 14 is a chart showing thermal relaxation time vs. pulse energy for a given fluence of 2.5 J/cm2.

FIGS. 15A and 15B illustrate schematically raster scanning vs. interlacing of an area of skin to be treated in accordance with the present invention. FIG. 15A shows standard raster scanning with time T between adjacent lines. FIG. 15B shows an interlacing scan by skipping K lines and returning after bottom line to the top. Time between rows is T*K.

FIG. 16 is a cross-sectional side view of a holder for a scanning head in accordance with a second embodiment of the present invention.

FIG. 17 is an exploded view of a separator assembly which forms part of the holder of FIG. 16 and is capable of testing scanner, laser and optical alignment prior to treatment.

FIG. 18 is a flow chart which illustrates operation of the holder to perform a test routine.

FIG. 19 is an illustration of an automated scanning work head connected to a balanced articulating arm positioned over a treatment chair in accordance with a third embodiment of the invention.

FIG. 20A is a schematic drawing of an underside of the scanning work head of FIG. 19.

FIG. 20B is a side view of the scanning work head of FIGS. 19 and 20A, including a schematic drawing of a subject with pigment to be removed.

FIG. 21A to 21E illustrate an example of an image acquisition, verification and tattoo-removal treatment sequence in accordance with the present invention. In FIG. 21A, an aiming beam indicates a scanning field outline to an operator. In FIG. 21B, a contour of a target area to be treated is measured and scanning parameters are calculated. FIG. 21C illustrates a preview of a scanning field/sequence using the aiming beam or an on-screen display. FIG. 21D and 21E illustrate the tattoo-removal sequence.

FIGS. 22A and 22B illustrate the effect of topography on the attainable scanning field. In FIG. 22A a flat target is shown and the whole scanning field of the scanner head can be used. In FIG. 22B, a non-flat target reduces the accessible scanning field. A spherical topology is shown for simplicity.

FIG. 23 illustrates schematically treatment of a pigmented area to be treated that is larger than an attainable scanning field. A stitching algorithm is used to treat the entire area in a plurality of scanning segments using pattern recognition of untreated areas with overlap.

FIG. 24 perspective view of a 6-axis robot mounted with a scanner head.

FIG. 25 is a schematic drawing of a dermatological treatment facility in accordance with a fourth embodiment of the invention in which a single pulsed laser beam from a single treatment laser can be selectively toggled between a plurality (in this case two) different treatment areas.

FIG. 26 is a schematic drawing of a dermatological treatment facility in accordance with a fifth embodiment of the invention in which a single high-energy pulsed treatment laser beam is split and steered in parallel into a plurality of different treatment areas.

FIG. 27 is a schematic drawing of a dermatological treatment facility in accordance with a sixth embodiment of the invention in which a single high frequency (pulse repetition rate) pulsed treatment laser beam is multiplexed into a plurality of separate rooms in parallel by pulse-picking.

FIG. 28 is a timing diagram for the pulse-picking used in the dermatological treatment facility of FIG. 27.

FIG. 29 is a schematic drawing of the electrical and electronic components and connectivity of a dermatological laser treatment apparatus according to the one embodiment of the present invention shown in FIG. 1.

### DETAILED DESCRIPTION OF THE INVENTION

### Example 1

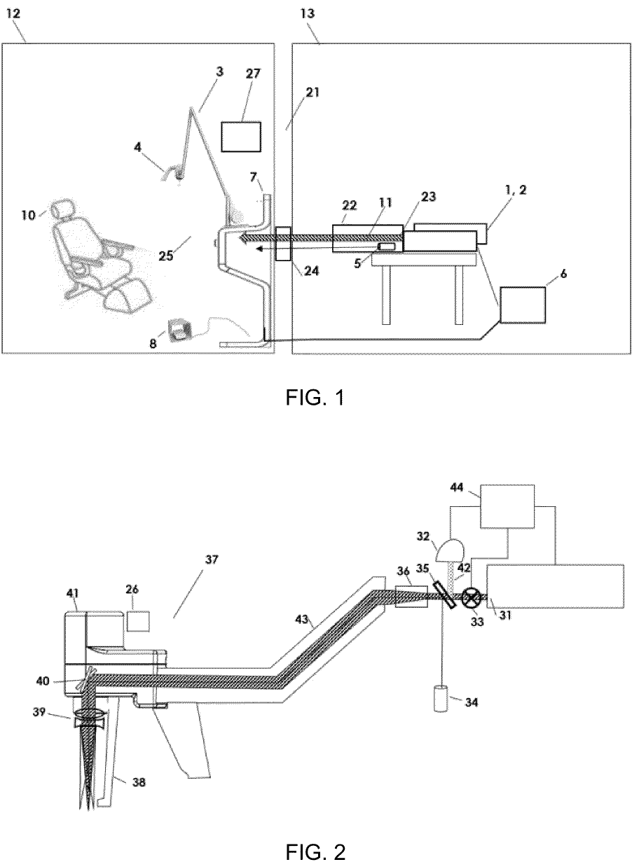

FIG. 1 of the accompanying drawings shows schematically a dermatological treatment facility in accordance with one embodiment of the present invention. The facility is provided in two adjacent rooms 12, 13 separated by a dividing wall 21. One of the rooms 12 is a treatment room; the other is a laser room 13 housing first and second treatment lasers 1, 2. In the present embodiment, the first laser 1 is a 800 nm Ti: Sapphire laser that produces ultra-short pulses, and the second laser 2 is a 1064 and 532 nm Nd:YAG laser. The Ti sapphire laser emits 100-30,000.00 femtosecond pulses, with 1-10 millijoule energies at 1 Khz pulse repetition rate. The Nd-Yag laser emits sub nanosecond pulses at similar energies and 500 Hz pulse repetition rates. It will be understood that different lasers may be used in other embodiments of the invention. An aiming beam 5 is coupled optically to the treatment lasers 1, 2 as described below to assist in placing a work head 4 as described in more detail below in a correct position over an area of a subject’s skin to be treated. A power and control unit 6 is provided, which includes a computer, power supply and dedicated controllers for system operation.

The laser room 13 ensures optimal conditions for the lasers 1, 2 are maintained. The treatment room 12 contains only operator and subject-accessible equipment.

Each of the first and second treatment lasers 1, 2 has a laser output 23 which is connected to an optical system 22 for directing laser beams 11 produced by the lasers 1, 2 through the dividing wall 21 into the treatment room 12 where they are supplied to a work station 25 comprising dermatological treatment apparatus in accordance with the invention. The optical system 22 may be any suitable arrangement of mirrors lenses and other optical components known to those skilled in the art (described below) and is received in a protective conduit where it passes through the dividing wall 21.

In the treatment room 12, adjacent the work station 25 there is provided a treatment chair 10 for a subject to be treated (not shown).

The work station 25 comprises a console 7 and an articulating arm 3 that is fixed to the wall or floor of the treatment room 12 for stability. The articulating arm 3 carries the above-mentioned work head 4 at its free end. The articulating arm 3 is capable of directing the treatment laser beams and the aiming beam optically (by using mirrors and joints assembly) into an optical input on the work head 4 at any point in a certain volume of the treatment room.

The work station 25 is connected to a foot pedal 8 for controlling laser output.

Referring to FIG. 2, the treatment laser 31 (previously referred to as lasers 1 and 2 in FIG. 1 ) is controlled by a dedicated controller 44. For clarity only one treatment laser 1 is depicted, but arrangement of the second laser 2 is similar. Each treatment laser output 31 is monitored in real time by a fast detector 32 which is operable to sample a small percentage of the beam 42 using a beam sampler 35 (which also serves as an aiming beam coupler in this embodiment). The controller 44 is configured to shut down the laser power and close a shutter 33 automatically in the event that the output of the treatment laser deviates from maximal or minimal pulse energy.

The aiming beam 34 is coupled to the treatment laser beam optical path by a coupling mirror 35. Beam path then travels through a beam expander 36 for propagation through the rest of the system. Travelling through the articulating arm 43, both beams arrive at the work head 37.

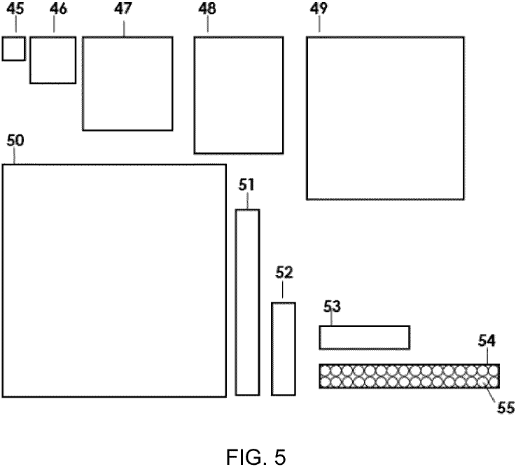

The work head 37 includes a detachable spacer 38 and a galvanometric scanner 41. In use, the laser beam travels into the galvanometric scanner 41 where it is directed by motorized mirrors 40 to pass through lenses 39 and onto a subject’s skin. The spacer 38 extends away from the work head and terminates in a smooth distal end for contacting the subject’s skin. The scanner galvanometric mirrors 40 are rotated so that the beam arrives at the focus lens assembly 39 at an angle. This angle is translated to a position on the subject’s skin through the focus lens assembly. The lenses create a desired spot size on the surface of the skin, which can be resized by an operator to attain required fluence. In the present embodiment a spot size of 0.7 mm is used for a 4 J/cm2 fluence, but it will be understood by those skilled in the art that any spot size of less than about 2 mm, preferably less than about 1 mm may be used, with a fluence in the range of about 0.5-50 J/cm2, preferably about 1-30 J/cm2. The scanner 41 then steers the spot across the skin within a scanning field of adjustable size and shape. The distance between adjacent spots is configurable and is typically less than about 0.1 mm. In some alternative embodiments, overlapping spots having a gaussian profile may be used. The amount of overlap may typically be about 0.1 mm. Different selectable rectangular scanning fields are shown in FIG. 5 by way of example. In particular, in the present embodiment, the galvanometric scanner 41 is operable to scan a configurable rectangle of from about 1 mm to about 10 mm in the length and/or width. It will be understood that in other embodiments, the scanning field may have any predetermined or arbitrary shape within the limitations of the scanner 41. The distance to the area of the subject’s skin may be determined by the spacer 38.

The work head also contains a motion sensor 26. During operation of the main laser, if the motion sensor detects motion above a predefined threshold it signals the control unit 6 to stop main laser immediately. This helps prevent unintended or uncontrolled lasing.

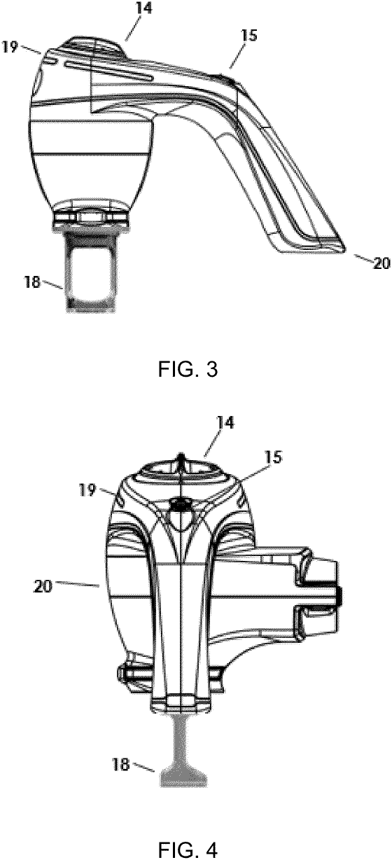

The work head 37 includes an outer shell 20 as best shown in FIGS. 3 and 4 which is designed as an ergonomic, plastic assembly that includes a scanning field size selector knob 14, an outline switch 15 to activate the aiming beam 34, indication lighting 19 and a replaceable spacer 18 (38 in FIG. 2).

The scanner mirrors are programmed to scan one of a set of predefined rectangles FIG. 5 45-54 in sizes ranging from 1 to 10 mm with various aspect ratios. Each rectangle corresponds to a specific setting of the field selector knob 14. In other embodiments of the invention the scanner mirrors may be operable to produce scanning fields of different sizes and/or shapes other than rectangles, e.g. circular scanning fields.

As best seen in FIG. 29, the controller unit 6 includes a power supply 401 with power lines 401, a processor 402 with memory for storing software and data 404, and real time controller 405, and programmable logic device 403. Combined, these assure smooth and safe operation of the system with acceptable redundancy. Signals and data are connected to various system components (1, 2, 5, 4, 37 etc) as shown through power and data lines 411. The real time controller 405 and processer 402 communicate with a digital scanner controller 406, which in turn operates an analogue scanner driver 407. Those supply both power and control to the galvo mirror scanner 41 in the work head 37. For clarity many connections and details have been omitted.

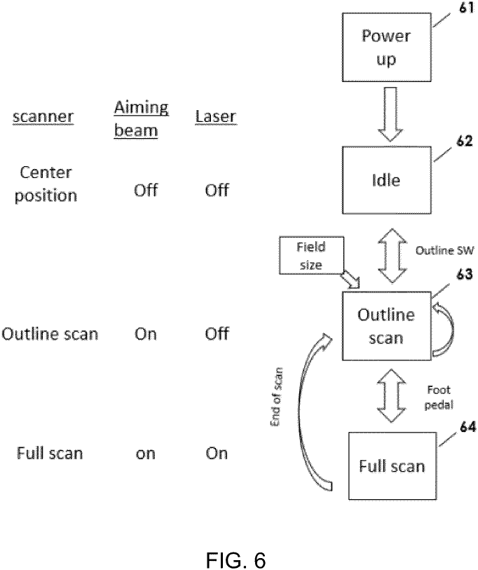

The system follows the logic depicted in the diagram of FIG. 6. During power up 61, several safety checks are performed, followed by the idle state 62. Once the outline switch 15 is switched on by the operator, the system moves to outline mode 63. The system remains in outline mode, continuously scanning the rectangle outline (i.e. one of 45-54) with the aiming beam 34, until the outline switch 15 is turned off or until the foot pedal 8 is depressed. Once the foot pedal is depressed, the main laser is turned on and pulses of laser light are scanned across the rectangle scanning area in a full scan mode 64. Once the entire area has been irradiated by laser pulses, the laser is switched off and the system returns to outline mode 63.

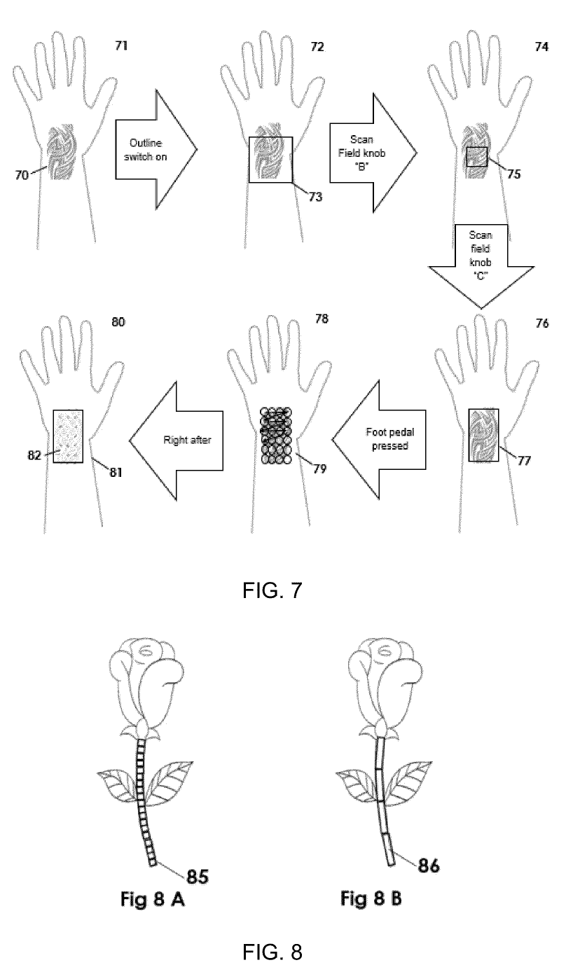

During application of treatment, the operator inspects the shape and size of the pigment to be removed 70. Once the operator turns on the outline scan the aiming beam then outlines the rectangle currently chosen on the subject skin 73 as shown in FIG. 7. This allows for visual feedback and accurate alignment of the scanner on to the treated area. Operator adjusts the field selector so that the rectangle fits the pigment shape and size and places the work head accurately over the pigmented area 77. Once the operator is comfortable with the set scanning field placement, the foot pedal 8 is depressed, and main laser then irradiates the subject’s skin by covering the entire area of the scanning field with laser spots -one spot in each location 79. In the present embodiment, the full scan duration is shorter than 1 second, typically 0.5 second. After the full scan, the main treatment laser is switched off and the aiming beam 34 outlines treated area again 81. Treated areas are usually visible due to a frosting effect of pulsed laser and skin interaction 82. To achieve optimal results, a laser set point is chosen as described in Example 2 below.

The throughput benefit of adapting the scanning field shape to the tattoo or other area of pigment can be appreciated by comparing the examples shown in FIG. 8A and B. To cover an elongated shape such, for example, as a stem of a rose, using appropriate rectangles 86 requires five scans. For a square or circular shape 85, the number of scans is about 3x greater. Since a single field scan is very fast, the placement process of the operator is the main contributing factor for total treatment time. Thus 3x fewer fields translates to ~3× faster treatment time. For a 3 mm thick and 50 mm long stem, for example, using a 500 Hz pulse repetition rate and a 0.6 mm spot size with no overlap, we arrive at approximately 830 spots, which equals 1.7 seconds of net scanning time. Using a 3x3 mm field (see FIG. 8A), there are approximately 17 separate fields to scan. Assuming a well-trained operator with a field placement time of the order of 0.75 second, we have 12.75 seconds of placement time and 14.45 seconds of total treatment time. When using a rectangular field of 3x10 mm as shown in FIG. 8B, we have ~5 placements, so total treatment time is 5.45 seconds. Although the difference between 15 and 5 seconds is not prohibitive for a treatment session, by repeating this analysis for much larger pigmented areas with complex shapes and features, it is evident that by optimizing the field shape, a huge reduction in treatment time can be achieved. Example 5 below takes this concept further to achieve even shorter treatment times.

### Example 2

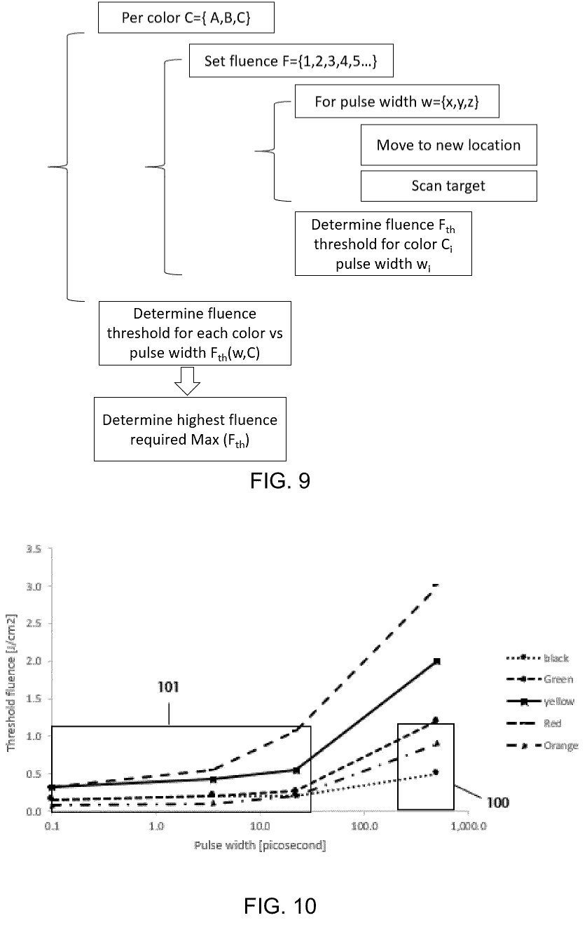

Using ultra-short and ultra-high intensity radiation in accordance with the present invention is beneficial for removing several colours with one wavelength, beyond linear absorption which is highly colour selective. When designing a new system one must determine the proper laser working point in order to achieve multi-colour pigment removal. A working point comprises fluence, pulse width and intensity. The intensity is required to be high enough for multi-colour removal, and it is usually determined by the combination of fluence (energy density) and pulse width. Fluence should be high enough to support the intensity, but not too high as to create excessive damage (typically about 0.5-10 J/cm²). Pulse width should be short but is usually limited by the specific laser design. Preferred pulse widths are of the order of about 0.5-30 picoseconds. Pulse energy is discussed below in Example 3. The optimal working point depends on the wavelength of the specific laser, the target colours (the more the better, usually) and the available laser pulse width of a specific laser system. To find a proper working point, we measure the reaction threshold of different ink colours/skin pigments in a lab setup. The test is repeated for each target pigment. A test target is created by mixing Gelatine, water and pigment. Referring to FIG. 9, the target is then scanned with a set of fluences, where for each fluence, pulse width is modulated (in effect modulating intensity). Once interaction is witnessed in the target (usually according to damage in the target) the threshold fluence for a specific pulse width is determined. By finding the highest intensity needed for the hardest to damage colour, we have arrived at a required intensity that covers all colours. It should be noted that in this method, intensity and fluence are tested independently though pulse width modulation, and is inherently different from prior art methods, where fluence and intensity are coupled since pulse width is constant.

In FIG. 10 shows an actual lab measurement result. As pulse width increases, more and more fluence is required for effective interaction with a target pigment. The spread of different fluences required for different colours also increases considerably. Prior art laser systems typically work at >250 ps pulse width and lower intensities. For a given fluence at pigment location in the tissue of say ~1 J/cm2 (indicated by reference numeral 100 in FIG. 10), a system might be able to remove green and black for example, but would not be able to remove red and yellow colours. This is because yellow and red have a considerably higher fluence threshold for that pulse width. Alternately one can state that “they do not absorb enough” in the given wavelength and pulse width. By decreasing the pulse width below ~25 ps in accordance with the present invention, the same fluence 101 is capable of removing successfully all colours in this case. This is because at this pulse width, the threshold for interaction is below the given fluence. It should be noted that the same wavelength as in the prior art is now capable of removing all colours as a result of the greater intensity.

The above method is applicable per specific laser wavelength, where different lasers require different maximal intensities and/or fluences depending on target colours vs. used wavelength. But once the threshold intensity is used, all target colours will be removed by that specific laser.

By “removal” herein it is intended that full clearance of colour from skin (to naked eye) may be achieved after a finite number of sessions. The number of sessions might vary from one target colour to the next, but in any case, the number of sessions from one colour to the next will not vary by more than a factor of about two.

Commercial lasers are available for the prescribed parameter set. See for example PicoLaser ltd “Pico-1M” laser with 8 mJ and 8 ps pulse width, or Amplitude Laser ltd “Magma” laser with 30 mJ and 1.5 ps pulse width.

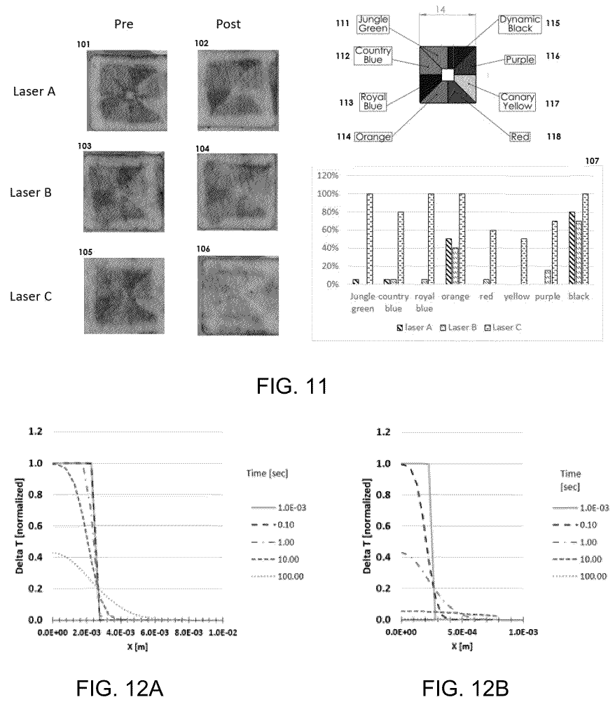

FIG. 11 depicts actual removal results in a controlled experiment performed on live porcine skin, as an example of the above stated method and system. The target is a multi-coloured square, including areas coloured green 111, blue 112, cyan 113, orange 114, red 118, yellow 117, purple 116 and black 115. Middle of target is untattooed, while boundaries are outlined black.

Using various pulse width and several treatments in the span of two months, pre- and post-images are shown 101-106. Laser A used a pulse width of 6 ns; laser B 0.6 ns; and laser C employed 1-15 picosecond pulse width (100-1000x shorter). Lasers A, B used 4 J/cm2 fluence, while Laser C used 2 J/cm2 fluence;. The intensity of each of lasers A/B was 0.7 /7 GW/cm2, while laser C had an intensity above 50 GW/cm2. With lasers A, B noticeable removal is achieved in outline black (101 vs. 102 and 103 vs. 104). For the short pulse laser C, all tattoo colours responded and above 80% clearance was achieved (105 vs. 106). Quantitative clearance levels are shown at 107.

### Example 3

The process of laser pigment removal, although targeting pigment, creates local heating in the tissue surrounding the pigment. Although local damage in tissue holding pigment is unavoidable, the surrounding tissue, not directly damaged by pigment radiation absorption, will suffer from secondary heating. The duration of local elevated heating is at the root of higher damage to surrounding tissue. In the following example we will quantify these effects.

During irradiation of pigmented tissue, the following occurs: Initially, on the time scale of laser pulse width, radiation is absorbed in parts of the tissue that are absorbing, usually in specific chromophores that are targeted for treatment. These can achieve very elevated temperatures (even thousands of degrees) in very short time scales of pico or nano seconds. This usually leads to plasma creation, mechanical breakdown and/or other violent events, which are usually the desired effect of the treatment. Nevertheless, after a short time, all this energy is converted finally to heat: plasma radiation is re absorbed after pulse is over, kinetic particles are slowed through collisions to a halt. Except for chemical alteration (usually an undesired effect), eventually all of the incoming radiation is converted to heat.

For time scales much longer than pulse width we can use a bulk heat approximation for absorbing layer to estimate the temperatures induced in the tissue. Considering, for example, an average 2.5 J/cm² fluence, 500 mJ pulse energy and a 5 mm spot diameter. Tattoo ink, for example, which absorbs laser radiation, is usually/predominantly at a depth from 300 to 700 µm below skin surface. We assume all radiation is absorbed in that thickness, within a cylinder of diameter as of the input pulse (for simplicity) and use water specific heat as a good estimation. Using ΔT=E/M•C we arrive at a temperature elevation of approximately 15° C. above ambient skin temperature of ∼34℃ (outer) to 36.8° C. (inner)

| | | |
|---|---|---|
| Water specific heat [C] | 4.18 | J/gr/deg C |
| specific weight | 1000 | gr/liter |
| spot diameter | 0.50 | cm |
| pulse energy [E] | 500 | mJ |
| absorption depth | 0.04 | cm |
| fluence | 2.5 | J/cm^2 |
| volume | 8.00E-03 | cm^3 |
| Mass [M] | 8.00E-03 | gr |
| ΔT | 15 | Deg cell |

This is also true for a 5 mJ pulse energy with 0.5 mm diameter (100x lower energy and 10x smaller diameter). The same average heating will always occur in this approximation if fluence is similar.

The advantage of applying only small, low energy pulses is clear by looking at heat diffusion over time. After a very quick initial heating process (in the order of nanoseconds or less), heat starts to diffuse away from the initial heated volume. Considering that the subject body is an infinite heat reservoir compared to the total pulse energy even in the high pulse energies, the diffusion will gradually reduce the temperature of heated volume back to natural body temperature. The rate of this cooling effect depends greatly on the volume of the heated tissue, which is very different in the above examples. More precisely, the rate is determined by the ratio of volume to surface area of the heated tissue. A small volume will cool down much more rapidly than a large volume.

To quantify relevant time scales, consider the case of two pulses with same fluence as in Example 2 above. Assuming just one pulse hitting the tissue, at what rate will the heat diffuse? Solving the linear heat diffusion provides us with the radial profile of the temperature at different times after initial heating at time t=0. Temperature profile of high energy, large spot (FIG. 12A) and low energy, small spot (FIG. 12B) are shown in FIGS. 12. After about 1 second, there is only a minor change in elevated temperature for the 500 mJ pulse 121, while the 5 mJ pulse temperature has dropped by approximately 50% 122. For 500 mJ pulse, it takes in the order of 100 seconds for the temperature to drop by 50%.

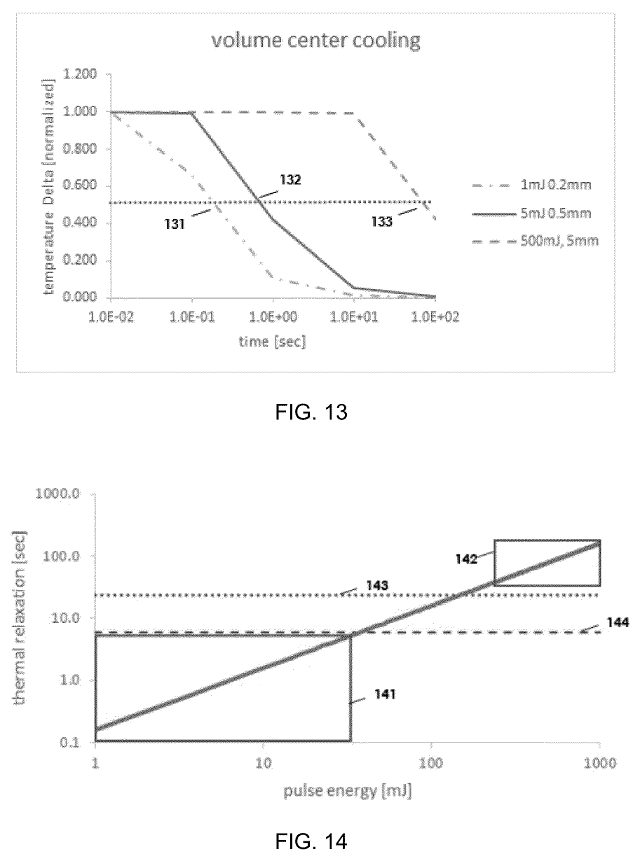

Thermal relaxation time may be defined herein as the time at which the temperature delta has dropped by a factor of 2x. Temperature of centre of heated tissue volume as a function of time is plotted in FIG. 13. For a 1 mJ pulse with 0.22 mm spot, thermal relaxation time is about 0.12 seconds. For a 5 mJ, 0.5 mm spot, relaxation time is about 0.7 s, while for a 500 mJ, 5 mm spot relaxation time is about 70 s.

FIG. 14 is a chart showing thermal relaxation time vs. pulse energy for a given fluence of 2.5 J/cm2. Box 142 shows a working point according to a prior method using 200-1000 mJ pulses. Relaxation times of 30-200 seconds are typical. In box 141 much shorter relaxation times of 0.1-8 seconds are provided in accordance with the invention using smaller pulse energies of 1-30 mJ. Skin damage thresholds are plotted in 143,144.

Given that the skin can withstand only about 6 s at 51° C. before damage occurs as discussed previously 144, it is thus clear in the above example, using a 5 mJ pulse, the skin can sustain a 15° C. temperature elevation to around 51° C., since it is dissipated in less than 1 second. For a 500 mJ pulse, with the same temperature elevation, damage will occur since the relaxation time is about 70 s, much longer than the damage threshold. The same analysis is true for skin temperature of 50 degrees, which can be tolerated for 24 seconds before damage occur. It is also known that pain appears before damage occurs. The pain threshold is lower than the damage threshold, but the temperature dependence is similar (Yarmolenko).

For this reason, pain and damage are both reduced or completely avoided by using small energy pulses (1-30 mJ) instead of large pulses above 200 mJ.

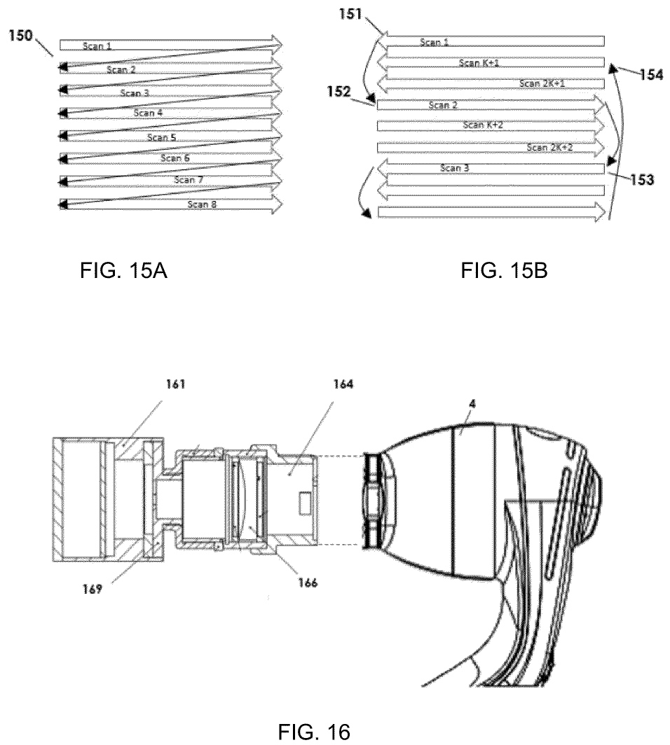

The above calculation reflects a comparison of high energy pulse to low energy pulse, at the same fluence. In order to gain for the benefit of fast relaxation time when scanning a large area with multiple spots, it is important to provide adequate time between adjacent pulses. This can be achieved by employing dedicated scanning techniques. As an example of smart scanning technique to increase available relaxation time for adjacent spots is shown in FIG. 15B. In a normal raster scan of N lines (FIG. 15A) each thick line 150 is composed of multiple spots. The time it takes to complete a line is T. This means that after time T, each spot will have a new neighbour below and this is in addition to its neighbours left and right in its own line. Now lets us use an interlacing scan (FIG. 15B): this means that instead of scanning lines continuously, we scan the top line 151 and then skip M lines down to mark the next line 152 much further away. We continue this until we reach the edge of the scanning field, were we return to the second line from the top 154 and repeat the process. This gives adjacent lines a relaxation time of K*T, K=floor(N/M) which can be potentially much longer for larger fields.

### Example 4

In order to assure proper functionality of the system and safety of subject and operator, the system in Example 1 may be adapted to include specialized test hardware and sequence in a dedicated work head holder which may be located in the treatment room 12. The holder 27 of the invention includes a work head interface 164, optical lenses 167, a perforated separator 169, and an optical power meter 161.

Referring to FIG. 16 the work head 4 is connected to the right side of the holder 164 as shown in the drawing. It should be noted that the connection is identical mechanically to the connection of the spacers 18 as described above with reference to FIG. 3 used for treatment.

To the left of 164 is a lens 166 for adapting the optical distance to the power meter 161. Above the power meter there is a separator 169, were several holds have been drilled to allow the laser radiation to reach the power meter.

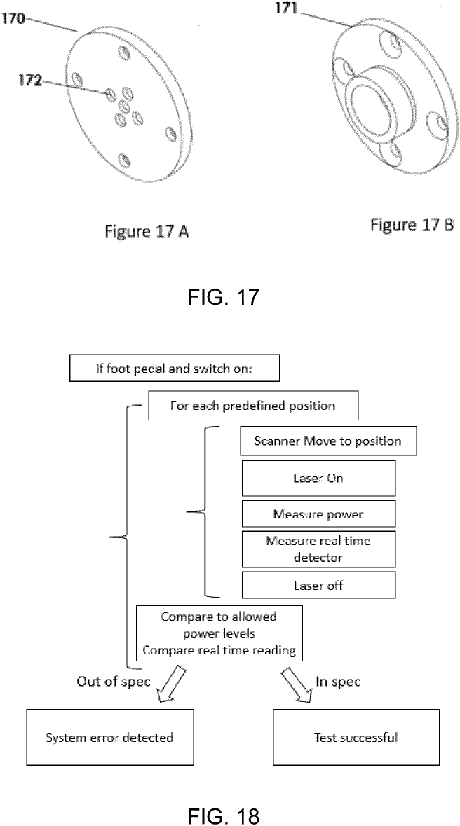

FIG. 17A shows the separator left side 170 in the direction of the detector and also an arrangement of several holes 172 that extend through the separator. FIG. 17B shows the right side of the separator 171 in the direction of the work piece.

The test sequence is shown in FIG. 18. The sequence starts only when the proper controls have been applied, mainly aiming beam switch 15 on and food pedal 8 is pressed. The scanner mirrors 40 are then moved to positions corresponding to hole positions in the separator 172. In each position, the main laser ½ is turned on, and power is measured in the power meter 161.

Once all predefined positions and power measurements are performed, the measured power is compared to a predefined table with allowable ranges. Test is successful if all measurements are in the predefined ranges.

Several favourable aspects should be noted. The first aspect is that the power meter 161 (or any other relevant sensor) is located in a plane that is optically equivalent to the plane of the treated skin using a spacer 18. This is in contrast to prior systems where the laser radiation is usually measured closer to the laser output and not at the output of the system. This ensures that subject receives exact radiation parameters, and accounts for failures occurring anywhere along the optical path: from inside the laser, through the optical elements, the scanner and the lenses in work head (see FIG. 2).

Second, the different separator holes in different positions require the scanner mirrors 40 to arrive to predefined locations. This ensures the scanner mirrors, their actuators and their control electronics are all performing as expected. It also ensures that the optical beam has not wandered out of alignment in the angle or position, which will correspond to partially or completely missing the separator holes (just like a mirror actuator failure) and resulting in low power measurement.

Also, by creating holes of different diameters, the divergence of beam can also be accounted for. This divergence will result in different power levels measured compared to predefined ones in holes of different diameter.

In addition, during the test, real time sensors located next to laser output 32 (see FIG. 2) are compared to the holder sensor, insuring they are consistently measuring pulse energy.

Finally, the sequence requires operation of user controls in the same manner as the normal operation during treatment, and accounts for any failures in switches or controls.

It will be understood that the test apparatus does not necessarily need to incorporate a holder for the work head. In other embodiments, the sensors may be mounted to a supporting structure that is not designed to hold the work head as such, but has a work head engaging portion that is configured to engage the work head to locate it stably relative to the sensors for testing. Instead of the perforated separator as described above, the sensors may include at least one position sensitive detector for detecting the position and power of the laser beam.

### Example 5

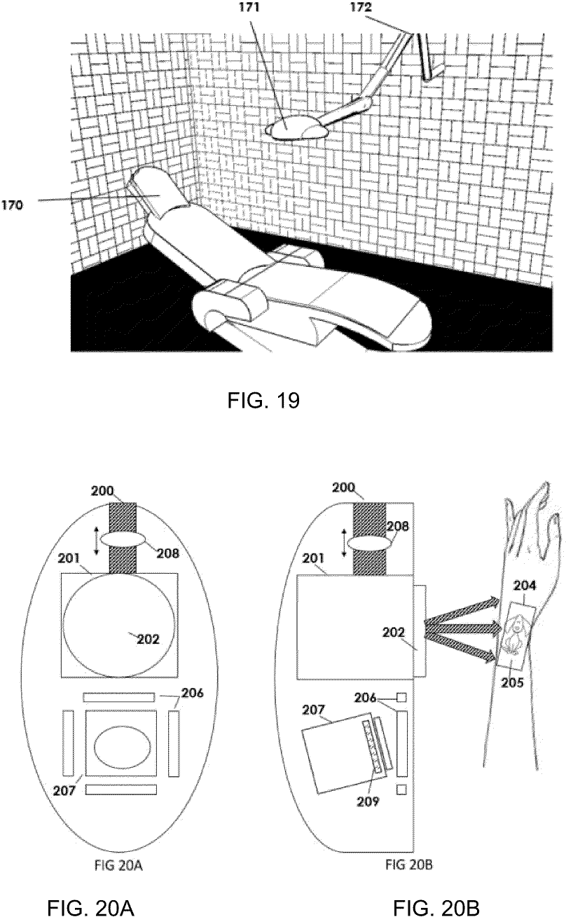

FIG. 19 illustrates a treatment room of a dermatological treatment facility which includes laser treatment apparatus adapted for automated scanning of an area of a subject’s skin to be treated according to another embodiment of the present invention. Above a treatment chair 190, a large optical work head 191 is suspended through a balanced articulating arm 192. The apparatus in a laser room (not shown) is similar to the apparatus described in Example 1 above, but the treatment room work head in the present embodiment is larger and utilizes imaging and other sensors to scan automatically a large area to be treated (compared with manually scanning small areas in Example 1).

Laser scanning is widely known from industrial material processing applications. As opposed to industrial application of laser scanning, where the same target material and sample are scanned repeatably in large quantities, in the present invention, however, a subject is only scanned once (at least per treatment), and a required scanning pattern is very rarely similar, as no two subjects and no two lesions are ever identical. Additionally, the cost of error is unacceptable and safety considerations are paramount. The following description shows how these complications may be addressed in accordance with the present invention to provide fast, accurate and safe scanning of lasers for treating dermatological indications.

In FIGS. 20A and 20B, components of the working head 191 are shown in two cross sectional views. Laser radiation enters the work head input 200 from one or more treatment lasers in the laser room through the articulating arm 192 and passes a motorized adjustable focusing lens 208. It then enters the scanner 201. This scanner is larger than the scanner in Example 1 and directs the laser beam through a 160 mm f-teta lens 202 to cover an area of 100x 100 mm on the subject’s skin 204 which is typically maintained at a constant distance of about [DISTANCE] from the work head 191. Scanner, integrated focusing and f-theta lens are readily available for example from ScanLab Germany or Cambridge technology MA, USA. A camera 207 mounted in the work head is operable for imaging the treatment area, while illumination LEDs 206 supply specific illumination conditions. The camera 207 is also capable of 3D measurement of depth and in addition to imaging the area to be treated can generate a height map of the area. 3D cameras are readily available, for example RealSense from Intel, USA.

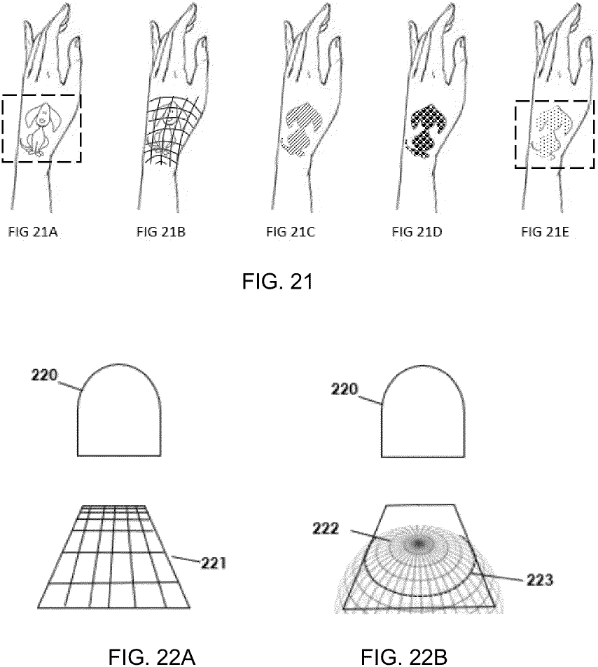

The treatment sequence is described in FIG. 21A-E . Initially, an operator manipulates manually the work head 191 to be placed roughly above the target area. The articulating arm is balanced such that there is little friction and operator can easily manipulate the work head. An aiming beam shows an available scanning field by outlining it (see FIG. 21A) to help the operator position the centre of the available scanning field to coincide approximately with the target area. Using image processing, the system then detects the pigmented areas based on images of the area captured by the camera 207. The 3D camera also measures the contours of the target area and scanning parameters are calculated (FIG. 21B). Once a scanning plan is defined, the operator is shown the planned area to be treated. This can be done with a dedicated computer interface but in this example it is shown directly on the subject target area: using only the aiming beam, the exact planned pattern as will be performed by the main treatment laser is scanned repeatedly (FIG. 21C) . The operator then approves the scanning plan by pressing a button in a user interface screen and then the work head scans the approved area with the treatment laser (FIG. 21D). Following this scan, the system returns to outlining the available field while treated area typically appears white as a result of frosting as described above (FIG. 21E).

It should be noted that all the pigmented skin within the scanning field is treated at once, without further operator involvement. This ensures accuracy of laser treatment while achieving very fast treatment time compared to manual placement (up to 40 seconds for a 100x100 mm tattoo, typically much less).

The system may use the premeasured and real time depth data to adjust the focusing lens 208 in order to account for scanner-skin distance and contours of skin surface. In some instances, the contours of the target area may be curved such that scanning the entire area is not possible: a bracelet tattoo around the wrist, for example. During the depth measurement (FIG. 21B), a pigmented area that is too curved to be treated (due to angle above 20 degrees or due to depth that is beyond the focus range of the system, typically 35 mm) is omitted from the planned scan. FIGS. 22A and 22B illustrate an example of this feature: in FIG. 22A a flat target is scanned, and the entire available scanning field 221 can be utilized. In FIG. 22B a curved surface below the scanner 220 implies that a smaller area of surface 223 can be scanned as compared to the larger area of the flat surface 221.

An image processing algorithm for detecting pigmented areas to be treated may be divided between tattoos, using first order derivative (sobol) operator for edge detection, while pigmented lesions with softer edges may utilize a trained neural network algorithm. These algorithms are easily understood by those skilled in the art. As accuracy of either algorithm is not 100%, the operator may use a suitable computer interface (not shown) to correct the algorithm results and manually adjust the scanning pattern if required. The pattern is then updated to the pre-scan (FIG. 21C).

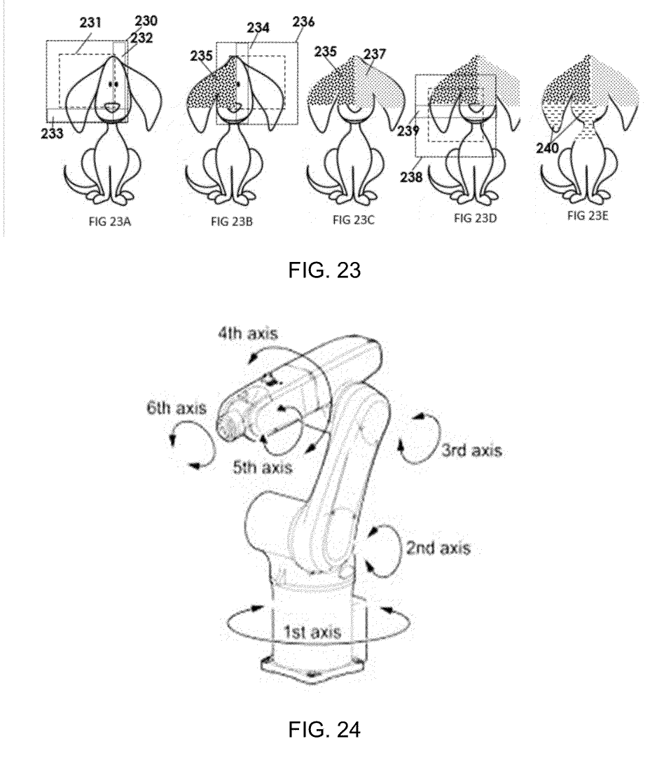

When treating an area larger than maximal scanning field or a curved area that cannot be treated in one scan, the treatment may be divided into several segments. The operator manually positions the scanner above each segment and starts the pattern recognition algorithm. Based on previous images compared to current image a suitable stitching algorithm identifies previous segments that were treated and so avoids treating areas twice or missing some areas. This algorithm is shown in FIG. 23A-E. In a first step (FIG. 23A), a scanner is placed above a top-left region of an area to be treated. The camera 207 captures area 230, which is larger than scanner maximal field 231. A pattern recognition algorithm identifies the pigmented area and treatment is then administered to this area 235. Scanner is then moved rightwards as shown in the drawings by the operator (FIG. 23B). The operator needs to verify that there is some overlap in new camera image 236 with previous camera image 230. This overlap is explicitly shown in 234 and 232 (previous and current image overlap area). Using this overlap, images 231 and 236 are stitched, and a new treatment area is now identified by the pattern recognition algorithm, but the area already treated in previous step 235 is masked. Thus the new scan area 237 is defined and scan is performed with no overlap with the previous scan (FIG. 23C). The operator then moves the scanner to the middle-left of the area (FIG. 23D). This time an overlap is found with area 233 of the first image compared to area 239 in new image. The stitching algorithm defines area 240 for treatment and scan is performed (FIG. 23E). It should be noted that the stitching algorithm relies on untreated skin, since treated skin is sometimes significantly different in appearance due to skin whitening (also referred to as frosting) as is frequent in laser treatment if skin.

When treating contoured areas, the above process may be repeated with an intermediate step of projection of a camera image into a flattened image using the measured curvature data. These algorithms are known to those skilled in the art. Combining large and/or contoured treatment areas is then straightforward.

Additionally, based on a predefined rule set (usually lasers for specific colours) pattern recognition algorithm identified specific pigment colours and recommends treatment laser wavelength.

During the main laser scan, which can take several seconds or more depending on tattoo size (see above), the subject may move. For this reason, the camera may continuous image the treatment area and monitor for movement. In order not to be blinded by reflection from the main the laser during scanning, a motorized optical filter 209 (see FIGS. 20) may be used during the treatment scan to block the various laser wavelengths.

The illumination sources 206 (FIGS. 20) are specially selected LEDs. Some of the LED may emit mainly visible range “white” light. These are used for pattern recognition of pigmented areas by the algorithm. In some embodiments, other LEDs may be specific to the UV range and/or others may be specific to the IR range. Several images may be taken using different illumination sources. UV images extract information on various pigmentation in the skin, while the IR images are used to assess the absorption of the IR wavelength laser.

This system may be integrated with a 6-axis robot to perform the placement automatically (FIG. 24). This may further increase utilization and accuracy of the system.

### Example 6

As clinical experience shows, in a laser treatment session, there may be a minimum period of 10 to 20 minutes of subject preparation and post-treatment care. The actual net laser treatment time can be equivalent or much faster: approximately 20 minutes for tattoos of 200 cm2 area using the system in Example 1 above; less than about 2 minutes when using the system of Example 5 (200 cm2 is the most common tattoo area to be removed based on clinical experience). This implies low utilization of the laser and system which incurs lower return rate on investment.

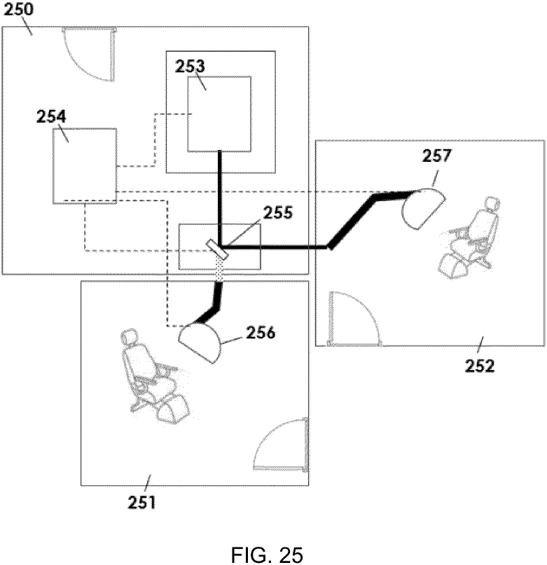

A solution to mitigate the above is a 2-treatment room facility in accordance with the invention, supported by a single treatment laser system. Referring to FIG. 25, the system includes one treatment laser system (with several wavelengths) 253, a control unit 254, two work heads 256,257 (e.g. as described in Example 1 or Example 5) in two treatment rooms 251,252 and a motorized flip mirror 255. Each treatment room also contains a treatment chair and all that is needed to administer treatment. In the present embodiment, the flip mirror, when in position, steers the laser radiation to the work head of room #1 252. When the flip mirror is out of the optical path, laser is directed to second treatment room and scanner head 256. The optical details and flip mirror are well known to those in the art and are not described in detail here. The control unit may be very similar to those described in Examples 1 or 5, with the additional control of a flip mirror. The work head is identical to the one described in the previous examples: articulating arm, scanner etc.

As one subject is being treated in the first room 252, a second subject may be prepared for treatment in second room 251. The control unit 254 is operable to accept operator commands from the first room work head 257 while commands from second room work head 256 are ignored. Once treatment is finished in first room (signalled by an operator turning off the work head), the controller toggles the flip mirror and diverts its control commands to be received from second work head 256. The subject in second room, now ready for treatment, starts treatment, and the first subject in first room may receive post-treatment care. The first room is afterwards cleaned, and next subject is prepared for treatment, so he/she is ready for treatment once the subject in second room finishes treatment.

This facility layout improves utilization by approximately a factor of two for either work head of Examples 1 or 5. For a system like the one of Example 1, with a 20-minute average treatment time, utilization may be above 90% as overhead (pre- post- subject care) and treatment times may be similar. Using a scan head similar to the one of Example 5 (automated area scanning) comparatively low utilization still results, as the time the treatment laser is actually scanning is still low (about 4 minutes in every 20-30 minutes). This will be addressed in the following examples.

### Example 7

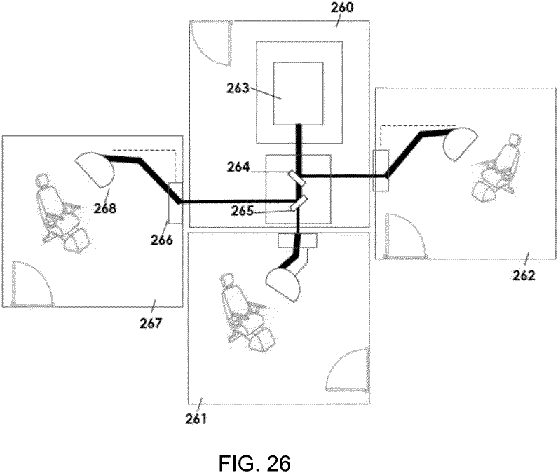

As discussed in Example 6, it is beneficial to increase overall utilization, lowered by subject pre-and post- care. The most expensive component in the system is the treatment laser. A system for increasing the system utilization by 3x is now described with reference to FIG. 26.

Using a laser capable of 3-4x higher pulse energy than is required for treatment (i.e. a laser capable of 10-150 mJ), the laser beam is split passively between three treatment areas 261, 262, 267 that are set up to work independently.

The laser 263 emits a powerful pulse which is split by a ratio of 1:2 in energy by a dedicated beam splitter 264. The smaller pulse (⅓ of the original) propagates to a first treatment room 262. The larger pulse (⅔ or original) continues to propagate in the direction of a second beam splitter 265, where it is split 1:1 and directed to a second room 261 and a third room 267 in parallel. Thus all three treatment rooms receive about 33% of original pulse energy.

In each treatment room, laser radiation is modulated independently according to control signals from each room separately. This may be achieved using a Pockels cell optical modulator 266 for room 267, for example. Thus three independent work heads are operational in three separate areas. The treatment laser 263 works continuously, and thus any optical modulator used at its output is not required. In effect, this modulator is actually placed in each of the three rooms. Unused pulses are dissipated in beam dump and the end of each optical modulator.

Thus the utilization of the laser is increased by 3x, at the cost of a more expensive laser and three dedicated optical modulators.

It will be self-evident to combine this embodiment with the previous one to achieve, e.g. a 6x improvement in utilization with a six-room facility.

### Example 8

In Example 7 above, a treatment laser with 3-4x higher pulse energy is split into three treatment rooms in parallel. Whilst scaling pulse energy is generally advantageous for reducing laser down-time, it may not always the best approach, as laser costs typically scale with pulse energy. In contrast, increasing pulse repetition rate while keeping same pulse energy (i.e. increasing average power) usually scales more favourably. This is because increasing average power involves (to a first order approximation) scaling pump sources and dealing with thermal load, while scaling pulse energy involves in addition dealing with laser induced optical damage to internal laser surfaces, which is mitigated by scaling beam area and thus increasing size and cost of optical components.

In the present example, a clinic supporting three treatment areas with a single laser is described. Here a laser of 3x higher repetition rate e.g. 600-3000 Hz but similar pulse energy 1-30 mJ (compared to a single room clinic laser) is used.

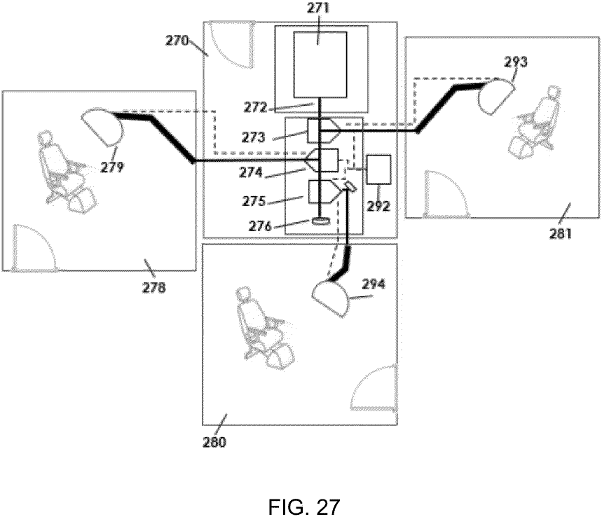

Referring to FIG. 27, a laser room 270 contains the above specified treatment laser 271, three fast optical modulators (Pockels cell) 273, 274, 275, a control unit 292 and a beam dump 276. The modulators are normally switched off, allowing laser output 272 to travel undisturbed to the beam dump 276. When one of the modulators 273, 274, 275 is turned on, all of the radiation is deflected by about 90 degrees in the direction of the corresponding treatment room. Radiation then reaches the work head in that treatment room.

The modulators are selectively turned on by a control unit 292 at one-third of the nominal laser frequency and have a phase of one cycle time between them, meaning a first one of the modulators can only opened once in every three pulses, a second one of the modulators can be opened only for the next pulse and then every 3rd pulse from the second, and so on. In effect, the modulators in combination are down-sampling the pulse train from the laser, with each treatment room receiving every the first, second or third pulse out of every three pulses.

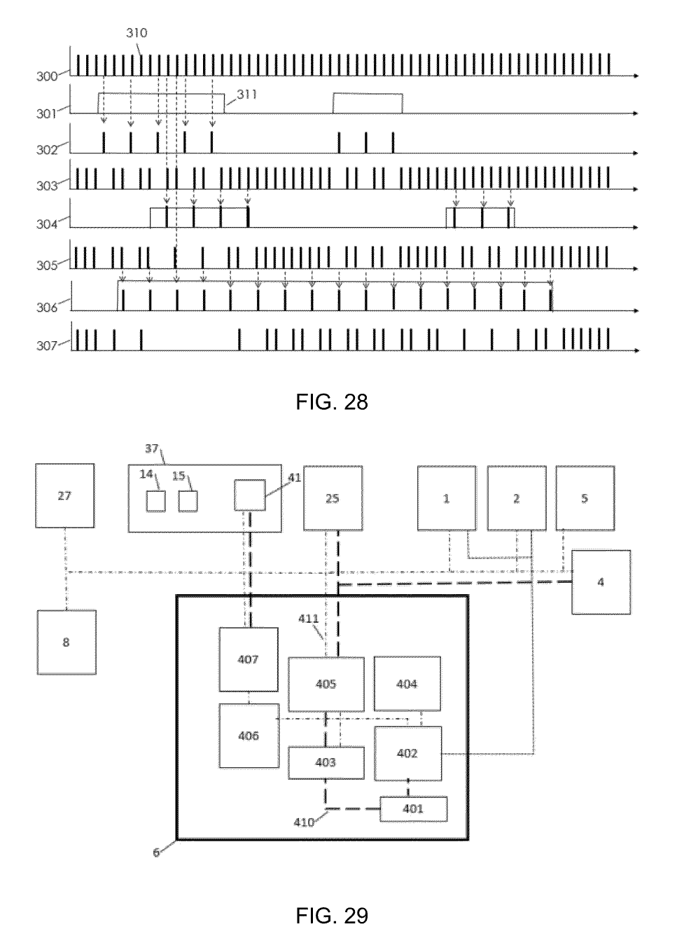

In addition to down-sampling, the modulators only steer the pulses when there is a demand for lasing from their corresponding treatment room. A detailed timing diagram is shown in FIG. 28. Laser pulses are depicted as dark rectangles 310, while the x axis represents time. The original pulse train from laser output 272 is shown at 300. Reference numeral 301 indicates a signal from the first treatment room work head, requesting a treatment scan at two separate time periods. At 302, the output of the first modulator 272, reaching the work head 292 in treatment room 281 is shown. Every third pulse from the laser is steered to the first room, when there is a request from the corresponding work head. The remaining pulses 303 continue in the direction of the second modulator 274. At 304, a requested treatment signal from the second room work head 279 is shown along with the pulses that are deflected by the second modulator 274 in the direction of the second treatment room 278 and eventually reach the work head 279. Undeflected pulses 305 continue toward the third modulator 275. Numeral 306 indicates a requested treatment signal of the third work head and the resulting pulses reaching the third room. Undeflected pulses 307 arrive eventually in the beam dump 276 where they are absorbed.

To summarize, three fast optical modulators utilize a high pulse repetition rate laser to treat simultaneously and independently three treatment rooms, thus achieving high utilization of the laser and creating a favourable return on investment. Whilst three modulators are used in the present embodiment to steer successive pulses of laser light to work heads three corresponding treatment areas, those skilled in the art will appreciate that in other embodiments, depending on the original pulse repetition rate of the laser, fewer or more modulators may be used to steer the beam selectively into two or four or more treatment rooms.

### Computing Devices and Systems

The computing devices and systems described and/or illustrated herein broadly represent any type or form of computing device or system capable of executing computer-readable instructions. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

Features from any of the embodiments described herein may be used in combination with one another in accordance with the general principles described herein. These and other embodiments, features, and advantages will be more fully understood upon reading the foregoing detailed description in conjunction with the accompanying drawings and claims.

The preceding description has been provided to enable those skilled in the art to utilise various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

### References

Anderson, R. R., & Parrish, J. A. (1983). Selective photothermolysis: precise microsurgery by selective absorption of pulsed radiation. Science, 220(4596), 524-527.
Goldman, M. P., Fitzpatrick, R. E., Ross, E. V., Kilmer, S. L., & Weiss, R. A. (Eds.). (2013). Lasers and Energy Devices for the Skin (2nd ed.). Taylor & Francis Group, LLC. Pierce County Emergency Medical Services. (n.d.). Disaster Burn Training. Retrieved May 30, 2019, from Pierce County: https://www.piercecountywa.gov/DocumentCenter/View/3352
Shannon-Missal, L. (2016, February 10). Tattoo Takeover: Three in Ten Americans Have Tattoos, and Most Don’t Stop at Just One. Retrieved from The Harris Poll: https://theharrispoll.com/tattoos-can-take-any-number-of forms-from-animals-to-quotes-to-cryptic-symbols-and-appear-in-all-sorts-of-spots-on-our-bodies-some-visible-in-everyday-life-others-not-so-much-but-one-thi/
Yarmolenko, P. S. (n.d.). Thresholds of thermal damage and thermal dose models. Retrieved May 31, 2019, from The International Commission on Non-Ionizing Radiation Protection (ICNIRP) : https://www.icnirp.org/cms/upload/presentations/Thermo/ICNIRPWHOThermo_2015_Y armolenko.pdf

1. A laser based system for medical, dermatological or ophthalmic treatment, in which the system includes:
(i) a laser located in a first part of a treatment facility;
(ii) a beam splitter or beam selector that receives laser pulses from the laser and directs the laser pulses to two or more treatment rooms or areas in a different part or parts of the treatment facility;
and the system further includes, in each of the treatment rooms or areas, a laser work head that receives laser pulses from the laser.

2. The laser based system of claim 1 in which the beam selector is configured to sequentially direct laser pulses to different laser work heads, each in a different treatment room or area.

3. The laser based system of claim 2 in which a beam selector directs laser pulses to one treatment room or area, and then to a different treatment room or area.

4. The laser based system of claim 2 in which the beam selector is a motorized flip mirror.

5. The laser based system of claim 2 in which the beam selector includes a set of modulators, such as Pockels cells, that are each selectively turned on by a control unit to down-sample the pulses from the laser and direct the down-sampled pulses to separate treatment rooms or areas.

6. The laser based system of claim 1 in which the beam splitter is configured to generate substantially simultaneous multiple laser pulses, each directed to different laser work heads, each in a different treatment room or area.

7. The laser based system of claim 6 in which the laser generates a single high frequency pulsed treatment laser beam that is multiplexed into a plurality of separate treatment rooms or areas in parallel by the beam splitter, configured for pulse-picking.

8. The laser based system of claim 1 which includes a series of two or more beam splitters or beam selectors.

9. The laser based system of claim 1 in which a beam dump is configured to receive laser pulses that are not sent to a laser work head.

10. The laser based system of claim 1 in which the laser is a femto-second laser, such as a 800 nm Ti:Sapphire laser.

11. The laser based system of claim 10 in which the laser emits 100-30,000 femtosecond pulses, with 1-10 millijoule energies at 1 Khz pulse repetition rate.

12. The laser based system of claim 1 in which there is a second laser, also located in the first part of the facility, that is not a femto-second laser.

13. The laser based system of claim 12 in which the second laser is a 1064 and 532 nm Nd:YAG laser that emits sub nanosecond pulses at 1-10 millijoule energies and 500 Hz pulse repetition rates.

14. The laser based system of claim 1 in which the first part of the facility is a laser room configured to ensure that optimal conditions for the laser are maintained.

15. The laser based system of claim 1 in which the laser has a laser output which is connected to an optical system for directing laser beams produced by the laser into the treatment rooms or areas where they are supplied to a work station comprising a work head.

16. The laser based system of claim 1 in which there are multiple treatment rooms or areas and each treatment room or area includes an articulating arm that is fixed to the wall or floor of the treatment room or area for stability, and a patient treatment chair.

17. The laser based system of claim 1 in which a dividing wall separates a laser room, housing the laser, from each treatment room or area.

18. The laser based system of claim 1 in which the system is configured to operate to sequentially treat patients in different treatment rooms or areas with the laser pulses.

19. The laser based system of claim 1 in which treatment room or area includes an articulating arm that terminates in a work head that includes a laser beam scanning system.

20. The laser based system of claim 19 in which each articulating arm includes an internal mirror system positioned at a joint to reflect a laser beam, generated by the laser, that is passed internally through the arm and to the work head to deliver laser pulses to skin or tissue to be treated.

21. The laser based system of claim 20 in which the laser beam is reflected at the internal mirror to pass through a limb of the arm, and when that limb rotates through an angle, then the mirror is mounted on a gear or mechanism that provides for the mirror to rotate through half that angle.

22. The laser based system of claim 1 in which the work head is configured to automatically scan or move the laser beam in a defined pattern and/or velocity across the skin or tissue to be treated.

23. The laser based system of claim 22 in which the work head is configured to be manually placed by a human operator at the correct region and the scanning head then automatically steers or moves the laser beam within that region.

24. The laser based system of claim 22 in which the work head steers the laser beam using galvanometric mirrors or any other beam steering method, such as acusto optical beam steering.

25. The laser based system of claim 22 in which the defined pattern is a scanning pattern over a tattoo or a region of a tattoo and the system is configured to remove that tattoo.

26. The laser based system of claim 22 in which the defined pattern is selected to ensure no significant thermal heating of the skin or tissue is achieved by the laser beam.

27. The laser based system of claim 1 in which a camera or imaging system displays the skin or tissue to be treated on a display or monitor, illuminated by an alignment laser that shows the perimeter, border or region to be treated by the laser.

28. The laser based system of claim 1 in which the work head includes a camera or imaging system that optically images the area to be treated and automatically enables the laser to be incident on the tissue to be treated only when the system automatically determines that the laser beam is correctly aimed.

29. A method of using a laser for medical, dermatological or ophthalmic treatment, comprising the steps of:
(i) providing a laser located in a first part of a treatment facility;
(ii) sending laser pulses from the laser to a beam splitter or beam selector, in which the beam splitter or selector directs the laser pulses to two or more treatment rooms or areas in the treatment facility;
(iii) providing, in each of the treatment rooms or areas, a laser work head that receives laser pulses from the laser and, in use, directs them to a patient.

30. A method of tattoo removal comprising the steps of:
(i) providing a laser located in a first part of a treatment facility;
(ii) sending laser pulses from the laser to a beam splitter or beam selector, in which the beam splitter or selector directs the laser pulses to two or more treatment rooms or areas in the treatment facility;
(iii) providing, in each of the treatment rooms or areas, a laser work head that receives laser pulses from the laser and, in use, directs them to a tattoo to be removed.
